(19) United States
(12) Patent Application Publication
(10) Pub. No.: US 2023/0411006 A1
(43) Pub. Date: Dec. 21, 2023

(54) METHOD FOR ASSESSING THE AREA UNDER THE CURVE OF AN IMMUNOSUPRESSANT AND ASSOCIATED METHOD AND SYSTEMS

(71) Applicants: INSTITUT NATIONAL DE LA SANTE ET DE LA RECHERCHE MEDICALE, Paris (FR); UNIVERSITE DE LIMOGES, Limoges (FR); CHU DE LIMOGES, Limoges (FR)

(72) Inventors: Jean-Baptiste WOILLARD, Limoges Cedex (FR); Pierre MARQUET, Limoges Cedex (FR)

(21) Appl. No.: 18/250,273

(22) PCT Filed: Oct. 26, 2021
(86) PCT No.: PCT/EP2021/079615

§ 371 Date: Apr. 24, 2023

(30) Foreign Application Priority Data
Oct. 26, 2020 (EP) 20306278.1

(51) Int. Cl. G16H 50/20; G16C 20/30; G16C 20/70; G16H 20/10

(52) U.S. Cl. CPC G16H 50/20; G16C 20/30; G16C 20/70; G16H 20/10

(57) ABSTRACT

The area under the curve (AUC) of an immunosuppressant is the best exposure marker for following the global exposure to an immunosuppressant in an organ of a subject. However, measuring AUC is complicated in routine care as it requires multiple blood samples during the dosing interval, which can be expensive and clinically impractical. The present invention proposes to use artificial intelligence technique to predict AUC values based on the value of some predictors. Such method conducts to surprisingly accurate results. Notably, the method enables to obtain satisfactory results with only two samples.

### TECHNICAL FIELD OF THE INVENTION

The present invention concerns a method for assessing the area-under-the-concentration-over-time (AUC) value at a specific time of an immunosuppressant in blood or plasma of a subject after administration of a dose of said immunosuppressant. The present invention also relates to a method monitoring patients carrying out the steps of a method for assessing. The present invention also concerns a computer program product and a computer-readable medium involved in these methods.

### BACKGROUND OF THE INVENTION

An immunosuppressant is a chemical compound that inhibits or prevents activity of the immune system of a subject. Such compound is also known as immunosuppressive drugs, immunosuppressive agents, immunosuppressants or antirejection drug.

Due to narrow therapeutic index together with a large inter-individual variability, a treatment implying immunosuppressant requests a therapeutic drug monitoring which consist in giving a personalized dose according to measure of the drug concentration at selected times.

It is therefore desirable to determine the best exposure marker among selected times or global exposure (AUC) to an immunosuppressant in an organ of a subject.

For instance, the article by M. brunet et al. entitled “Therapeutic Drug Monitoring of Tacrolimus-Personalized Therapy: Second Consensus Report” (Ther Drug Monit, volume 41, number 3, June 2019) is a discussion about the relevance of a plurality of exposure markers on the effect of tacrolimus, (a calcineurin inhibitor largely used for the prevention of rejection in solid organ transplantation).

In this article, two main markers are discussed to adjust the individual dose of tacrolimus: the trough level and the AUC.

The trough level (or trough concentration) C0 is the lowest concentration reached by a drug before the next dose is administered. The through level is largely used for practical and economic reasons although it has been inconsistently associated with outcomes.

By contrast, the AUC is theoretically the best marker of exposure while no formal proof has been obtained so far for tacrolimus.

Similarly, the analysis of the article by DR Kuypers et al. entitled “Immunosuppressive drug monitoring—what to use in clinical practice today to improve renal graft outcome” (Transpl Int., volume 18, number 2, pages 140 to 150, February 2005) shows that the best marker for therapeutic drug monitoring of ciclosporin is AUC while C0 is also largely used for the same reasons that tacrolimus.

For mycophenolate mofetil (MMF), AUC is consensually recognized as the best marker of exposure in the article of S. E Tett et al. entitled “Mycophenolate, clinical pharmacokinetics, formulations, and methods for assessing drug exposure” (Transplant. Rev. (Orlando) 25 pages 47 to 57, 2011).

Nevertheless, measuring accurately the AUC of an immunosuppressant is complicated in routine care as it requires multiple blood samples during the dosing interval, which can be expensive and clinically impractical.

To circumvent, such problem, various regression methods based on fewer blood samples have been developed but do not provide with an appropriate accuracy, while still requesting a relatively high number of blood samples.

### SUMMARY OF THE INVENTION

There is still a need for a method for assessing the AUC of an immunosuppressant in an organ of a subject after administration of a drug comprising a dose of the immunosuppressant, which is more convenient and accurate.

To this end, the specification describes a method for assessing the area-under-the-concentration-over-time value at a specific time of an immunosuppressant in blood or plasma of a subject after administration of a dose of the immunosuppressant, the subject being treated by a treatment comprising administrations of the drug, the method being computer-implemented and the method comprising the steps of providing parameters, the provided parameters comprising parameters relative to the treatment, and of applying a predicting function to the provided parameters to obtain area-under-the-concentration-over-time value at a specific time of the immunosuppressant, the predicting function being obtained by an artificial intelligence technique.

According to specific embodiments, the intelligence artificial technique does not comprise using a neural network. In particular, the intelligence artificial technique comprises using or is a gradient boosting technique, preferably an extreme gradient boosting technique.

The intelligence artificial technique does not comprise using a neural network.

It should also be mentioned that deep neural network approaches are associated with better performances than “classical” machine learning algorithms only when large samples are used. This is notably known from an article from Md Zahangir Alom entitled “A State-of-the-Art Survey on Deep Learning Theory and Architectures” Electronics 2019, 8, 292 (see notably FIG. 7).

This means that using an artificial intelligence technique other than a neural network enables to have fewer constraints on the learning dataset.

This is not natural for the person skilled in the art to consider a small dataset. Indeed, the person skilled in the art would rather consider augmenting the size of the learning dataset. Notably, in this context, the person skilled in the art would clearly consider using complete AUC curves and expects real improvement in the precision reached by the technique. The person skilled in the art could also use advantageously a technique to generate artificially new data by using the already available data. However, such techniques are not appropriate here.

Such property enables to use a specific way of constructing the learning dataset as will now be described.

The AUC have been extracted from the ISBA website (https://abis.chu-limoqes.fr. In brief, ISBA uses population pharmacokinetic models as prior by applying the Bayes theorem to estimate the individual AUC (posterior). This corresponds to a maximum a posteriori Bayesian estimation (MAP-BE). The MAP-BE estimation use only three concentrations+some covariates (time post transplantation, associated immunosuppressant, type of transplantation).

The preparation process of the dataset is, for instance, the following:
extraction of features and abbreviated AUC estimated from three samples in the ISBA website,
tidying the data and graphical exploration,
feature engineering: creation of difference in concentrations and relative deviations to theoretical time,
development of machine-learning algorithms based on two or three samples: tuning of hyperparameters by crossvalidation,
evaluation of performances using 10-fold crossvalidation,
evaluation in the test set by calculation of bias and imprecision, and
evaluation in external dataset based on full AUC (calculated from at least 8 samples) by calculation of bias and imprecision.

According to further aspects of this method for assessing, the parameters relative to the treatment comprise several values of concentration of the immunosuppressant at different times after administration of the drug, the number of values of concentration being preferably equal to 2.

According to another aspect, the parameters relative to the treatment comprise the values of difference in concentration of the immunosuppressant at a first given time and a second given time after administration of the drug for several couples of first given time and second given time.

Indeed, it appears the variables of difference between the concentrations sampled at the mentioned times is very important (just after the concentrations themselves).

These parameters are linked with the delayed peak observed in the administration of immunosuppressants. For instance, if the difference between the concentration at 1 h and the concentration at 3 hours is strictly superior to 0 μg/L for tacrolimus or mg/L for mycophenolic acid, there is no delayed peak while if the concentration at 1 h and the concentration at 3 hours is strictly inferior to 0 μg/L for tacrolimus or mg/L for mycophenolic acid, there is a delayed peak and that is correlated to AUC values.

In summary, on the one hand, the use of such parameters is very original and has not been used before and, on the other hand, the use of such parameters is very efficient for obtaining a robust prediction of the value of AUC.

According to further aspects of this method for assessing, the provided parameters further comprise at least one parameter relative to the subject, notably the age of the subject.

According to another aspect, the parameters relative to the treatment comprise the dose administered.

According to further aspects of this method for assessing, the treatment includes a transplantation of an organ to the subject, the parameters relative to the treatment comprising the delay between a request of assessment of the area-under-the-concentration-over-time curve of an immunosuppressant and the transplantation.

According to another aspect, the provided parameters consist in the parameters relative to the treatment.

According to further aspects of this method for assessing, the immunosuppressant is a calcineurin inhibitor, notably tacrolimus or ciclosporin.

According to another aspect, the immunosuppressant is an inosine monophosphate deshydrogenase inhibitor.

According to further aspects of this method for assessing, the immunosuppressant is a m-TOR inhibitor, notably sirolimus or everolimus

The specification further describes a method for monitoring patients, notably enrolled in a clinical trial, to provide a quantitative measure for the therapeutic efficacy of a therapy, notably the therapy which is subject to the clinical trial, by carrying out the steps of a method for assessing on said patients, the method for assessing as previously described.

The specification also relates to a computer program product comprising computer program instructions, the computer program instructions being loadable into a data-processing unit and adapted to cause execution of a method as previously described when run by the data-processing unit.

The specification further describes a computer-readable medium comprising computer program instructions which, when executed by a data-processing unit, cause execution of a method as previously described.

### BRIEF DESCRIPTION OF THE DRAWINGS

The invention will be better understood on the basis of the following description which is given in correspondence with the annexed figures and as an illustrative example, without restricting the object of the invention. In the annexed figures:

FIG. 1 is a schematic view of a system adapted to carry out a method for assessing, and

FIG. 2 to FIG. 7 are experimental figures.

### DETAILED DESCRIPTION OF SOME EMBODIMENTS

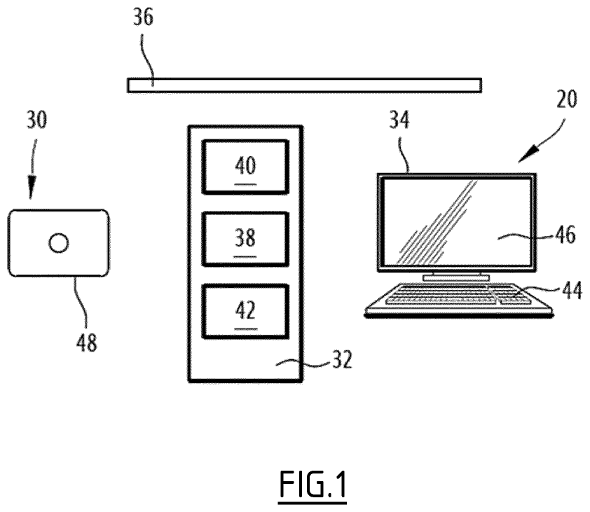

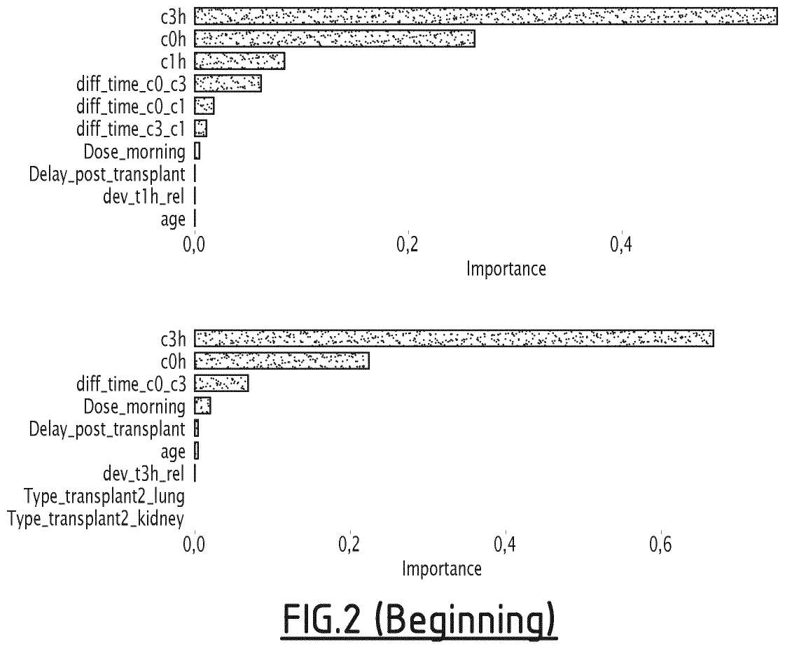

A system 20 and a computer program product 30 are represented on FIG. 1. The interaction between the computer program product 30 and the system 20 enables to carry out a method for assessing the AUC of an immunosuppressant in the blood or plasma of a subject as will be described later. Such method for assessing is named “assessing method” in the remainder of the specification.

This assessing method is a computer-implemented method.

The system 20 is a desktop computer. In variant, the system 20 is a rack-mounted computer, a laptop computer, a tablet computer, a PDA or a smartphone.

In specific embodiments, the system 20 is adapted to operate in real-time and/or is an embedded system, notably in a vehicle such as a plane.

In the case of FIG. 1, the system 20 comprises a calculator 32, a user interface 34 and a communication device 36.

The calculator 32 is electronic circuitry adapted to manipulate and/or transform data represented by electronic or physical quantities in registers of the calculator 32 and/or memories in other similar data corresponding to physical data in the memories of the registers or other kinds of displaying devices, transmitting devices or memoring devices.

As specific examples, the calculator 32 comprises a monocore or multicore processor (such as a CPU, a GPU, a microcontroller and a DSP), a programmable logic circuitry (such as an ASIC, a FPGA, a PLD and PLA), a state machine, gated logic and discrete hardware components.

The calculator 32 comprises a data-processing unit 38 which is adapted to process data, notably by carrying out calculations, memories 40 adapted to store data and a reader 42 adapted to read a computer-readable medium.

The user interface 34 comprises an input device 44 and an output device 46.

The input device 44 is a device enabling the user of the system 20 to input information or command to the system 20.

In FIG. 1, the input device 44 is a keyboard. Alternatively, the input device 44 is a pointing device (such as a mouse, a touch pad and a digitizing tablet), a voice-recognition device, an eye tracker or a haptic device (motion gestures analysis).

The output device 46 is a graphical user interface, which is a display unit adapted to provide information to the user of the system 20.

In FIG. 1, the output device 46 is a display screen for visual presentation of output. In other embodiments, the output device is a printer, an augmented and/or virtual display unit, a speaker or another sound generating device for audible presentation of output, a unit producing vibrations and/or odors or a unit adapted to produce electrical signal.

In a specific embodiment, the input device 44 and the output device 46 are the same component forming man-machine interfaces, such as an interactive screen.

The communication device 36 enables unidirectional or bidirectional communication between the components of the system 20. For instance, the communication device 36 is a bus communication system or an input/output interface.

The presence of the communication device 36 enables that, in some embodiments, the components of the system 20 be remote one from another.

The computer program product 30 comprises a computer-readable medium 48.

The computer-readable medium 48 is a tangible device that can be read by the reader 42 of the calculator 32.

Notably, the computer-readable medium 48 is not transitory signals per se, such as radio waves or other freely propagating electromagnetic waves, such as light pulses or electronic signals.

Such computer-readable storage medium 48 is, for instance, an electronic storage device, a magnetic storage device, an optical storage device, an electromagnetic storage device, a semiconductor storage device or any combination thereof.

As a non-exhaustive list of more specific examples, the computer-readable storage medium 48 is a mechanically encoded device such a punchcards or raised structures in a groove, a diskette, a hard disk, a ROM, a RAM, an EROM, an EEPROM, a magnetic-optical disk, a SRAM, a CD-ROM, a DVD, a memory stick, a floppy disk, a flash memory, a SSD or a PC card such as a PCMCIA.

A computer program is stored in the computer-readable storage medium 48. The computer program comprises one or more stored sequence of program instructions.

Such program instructions when run by the data-processing unit 38, cause the execution of steps of any method that will be described below.

For instance, the form of the program instructions is a source code form, a computer executable form or any intermediate forms between a source code and a computer executable form, such as the form resulting from the conversion of the source code via an interpreter, an assembler, a compiler, a linker or a locator. In variant, program instructions are a microcode, firmware instructions, state-setting data, configuration data for integrated circuitry (for instance VHDL) or an object code.

Program instructions are written in any combination of one or more languages, such as an object oriented programming language (FORTRAN, C″++, JAVA, HTML), procedural programming language (language C for instance).

Alternatively, the program instructions is downloaded from an external source through a network, as it is notably the case for applications. In such case, the computer program product comprises a computer-readable data carrier having stored thereon the program instructions or a data carrier signal having encoded thereon the program instructions.

In each case, the computer program product 30 comprises instructions, which are loadable into the data-processing unit 38 and adapted to cause execution of steps of any method described below when run by the data-processing unit 38. According to the embodiments, the execution is entirely or partially achieved either on the system 20, that is a single computer, or in a distributed system among several computers (notably via cloud computing).

Operating of the System

The operating of the system 20 is now described in reference to an example of carrying out an assessing method, which is a method for assessing the AUC of an immunosuppressant in the blood or plasma of a subject after administration of a dose of the immunosuppressant.

Such administration is mandatory in the case of organ transplantation from a donor to prevent organ rejection.

The term “donor” refers to the subject that provides the organ and/or tissue transplant or graft to be transplanted into the recipient.

This means that, in such case which is presented in this paragraph, the term “subject” designates the recipient that receives an organ and/or tissue transplant or graft obtained from a donor.

In this specific example, the subject is a human being.

More generally, the subject is a living subject and notably an animal.

For instance, the subject is a mammal, and more specifically a rodent such a mouse.

The subject is treated by a treatment comprising administrations of the dose.

Said treatment generally implies other medical acts, such as operations or drug administrations.

The method comprises a providing step and an applying step.

During the providing step, parameters are provided.

For instance, a user enters data in the input device 44.

Alternatively, the parameters are received by the system 20, notably from a remote server.

The provided parameters comprises parameters relative to the treatment.

As a specific example, a parameter relative to the treatment is several values of concentration of the immunosuppressant in blood or plasma at different times after administration of the dose.

Usually, 3 values of concentration are provided.

The first time corresponds to the initial time, which means inferior to 30 minutes and even most often inferior to 10 minutes.

The second time is generally set at 60 min more or less 50%. This value is the most advantageous.

In the examples that follow, the second time is set between 30 minutes and 100 minutes.

The third time is generally set at 180 min more or less 25%. This value is the most advantageous.

In the examples that follow, the third time is set between 140 minutes and 220 minutes.

It should also be noted that it is possible to obtain satisfactory AUC assessment with only two values of concentration.

Of course, having three values enables to obtain a better accuracy but it implies one more sample, which can be problematic in certain cases.

According to another example, a parameter relative to the treatment is the administered dose of immunosuppressant.

Generally, the dose of immunosuppressant is administered by oral route.

According to still another example, the treatment includes a transplantation of an organ to the subject, the parameters relative to the treatment comprising the delay between the request of assessment of the area-under-the-concentration-over-time curve of an immunosuppressant and the transplantation.

In the present example, the provided parameters consist in the parameters relative to the treatment.

According to other embodiments, the provided parameters further comprise at least one parameter relative to the subject.

For instance, a parameter relative to the subject is the age of the subject.

Thus, in a specific example, the provided parameter consist in the parameters relative to the treatment and the age of the subject.

Therefore, in the case of transplantation of an organ, the provided parameters may advantageously be:
2 ou 3 values of concentration,
the administered dose,
the delay between the request of the AUC assessment and the date of transplantation, and
the age of the subject.

During the applying step, a predicting function is applied to the provided parameters to obtain the AUC of the immunosuppressant.

The predicting function associates to the parameters provided as inputs an output which is the value of AUC at a specific time.

For instance, the AUC value at 12 h for a dose taken twice a day (TAD) or 24 h for a dose taken once a day (OAD) is of specific interest.

The predicting function is obtained by an intelligence artificial technique.

An artificial intelligence technique consists in establishing a model (also named algorithm) based on data.

In particular, the artificial intelligence technique often implies learning the model. The term “machine learning” is thus employed to designate the fact that the model is learned by the machine based on data.

According to the case, the machine learning technique implies using a learning among a supervised learning, an unsupervised learning, a semi-supervised learning, a reinforcement learning, a self learning, a feature learning, a sparse dictionary learning, an anomaly detection learning, a robot learning and association rules learning.

In particular, in the present example, the machine learning technique is a supervised learning technique, a semi-supervised learning technique or a reinforcement learning technique.

The model used in the artificial intelligence technique can be chosen from various models/algorithms, such as computational models and algorithms for classification, clustering, regression and dimensionality reduction, such as neural networks, genetic algorithms, support vector machines, k-means, kernel regression and discriminant analysis.

More generally, the artificial intelligence technique may imply the use of one or several of the following elements: sums, ratios, and regression operators, such as coefficients or exponents, biomarker value transformations and normalizations (including, without limitation, those normalization schemes based on clinical parameters, such as clinical data 58, gender, age or ethnicity), rules and guidelines, statistical classification models, and neural networks, structural and syntactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others.

Alternatively or in complement, the artificial intelligence technique may imply the use of one or several of the following elements: Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), boosting, regression analysis, Information fuzzy networks (IFN), statistical classification, AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

Alternatively or in complement, the artificial intelligence technique may imply the use of one or several of the following elements: artificial neural network, Data clustering, Expectation-maximization algorithm, Self-organizing map, Radial basis function network, Vector Quantization, Generative topographic map, Information bottleneck method, and IBSEAD, rule learning algorithms such as Apriori algorithm, Eclat algorithm and FP-growth algorithm, hierarchical clustering, such as Single-linkage clustering and Conceptual clustering, partitional clustering such as K-means algorithm and Fuzzy clustering.

Alternatively or in complement, the artificial intelligence technique uses a reinforcement learning algorithm. Examples of reinforcement learning algorithms include, but are not limited to, temporal difference learning, Q-learning and Learning Automata.

Alternatively or in complement, the artificial intelligence technique uses Data Pre-processing.

More specifically, the model is chosen among a linear model, a non-linear model, an ensemble model and a deep learning model.

A linear model is a model that uses linear relation(s) between the inputs and the outputs.

As a specific example, the linear model is penalized multinomial regression or linear discriminant analysis

A non-linear model is a model that uses non-linear relation(s) between the inputs and the outputs.

As a specific example, the linear model is a radial support vector machine.

A radial support vector machine is a classifier enabling to search a high-dimensional decision boundary to separate classes and maximize the margin.

An ensemble model is a model that aggregates multiple models to reduce loss.

In the present case, the ensemble model is an aggregation of several models and notably an aggregation of random forests (such algorithms aggregates concurrent multiple trees to reduce loss), gradient boosting machines (such algorithm corresponds to sequential and additive decision trees to reduce loss by using gradients in the loss function), extreme gradient boosting tree (this is an algorithm more efficient, flexible, and regularized than gradient boosting), naïve Bayes (it is a very simple and efficient probabilistic classifier. Naïve Bayes naively (strongly) assumes all features are independent).

Deep learning model is a model that uses multiple layers to progressively extract higher level features from the raw input.

As a specific example the deep learning model is a model averaged neural network.

Like random forest, model averaged neural network creates multiple neural networks to average them into one.

Advantageously, the artificial intelligence method is the artificial intelligence method that will now be described.

The artificial intelligence technique comprises a phase of preparing, a phase of training and a phase of evaluating.

During the phase of preparing, a data set is prepared.

The data set is formed by predictors and the AUC value.

In other words, the data set is a collection of data giving for many subject (for instance more than 100, preferably more than 1000) specific predictors and the AUC value of the immunosuppressant that was intaken.

The predictors of the data set encompass the provided predictors and usually additional predictors (feature engineering).

As an additional predictor, it can be considered the relative deviation from theoretical time and differences between concentrations.

In variant or in complement, other additional parameters are derived the provided parameters.

For instance, the difference in concentration between the provided values of concentration is of interest.

Thus, an example of predictor of a data set associates to the delay between the request and transplantation, the age of the subject, the dose of immunosuppressant, the immunosuppressant concentrations at time 0, a second time (1 h for instance) and a third time (3 h for instance) after dose intake, the relative deviation from theoretical time (see experimental section for a definition) and differences between concentrations, the value of AUC.

When only two concentration values are provided, each value implying the second time are not present.

During the splitting step, the data set obtained is split into a training set and a testing set.

For instance, ¾ of the predictors of the data set obtained at the end of the imputation step are considered as the initial training set, the other predictors being considered as the testing set.

Alternatively, ratios of 70/30 or 80/20 can be used at the splitting step.

It is to be noted that in complement, other operations can be carried out during the phase of preparing.

For instance, an imputation technique can be implemented. In statistics, imputation is the process of replacing missing data with substituted values.

As another example, the data set can be upsampled so that each kind of AUC values be represented.

If one imagines that the majority of AUC value in the data set is superior to a threshold, it may be interesting to artificially increase the number of elements with an AUC value inferior to the threshold.

As an illustration, the up-sampling comprises increasing the number of elements and iterating an operation of replacing such that the number of elements with an AUC value inferior to the threshold reach a predetermined value.

Standardization is an operation enabling that quantitative parameters be in a similar range which enables to foster the phase of training.

During the phase of training, the intelligence artificial technique comprises using a gradient boosting technique.

Extreme gradient boosting is a machine-learning approach based on boosting. In brief, simple regression trees are iteratively built by finding split values among all input variables that minimize prediction error. The iterative process constructs an additional regression tree of the same structure, but which minimizes the residual errors of the first regression tree.

In the described example, during the training step, heterogeneities are created in the set of data

For instance, in the current example, a k-fold cross-validation is repeated.

For instance, 10-folds cross-validation are randomly repeated three times with a new training process during which hyperparameters of the model are tuned.

Such creating step enables to minimize chance of overfitting and possible sampling bias.

Alternatively or in complement, the creating step comprises using bootstrapping.

During the tuning step, hyperparameters of the model adapted for controlling the training process are tuned.

Such procedure which is also named hyperparameter optimization finds a tuple of hyperparameters that yields an optimal model which minimizes a predefined loss function on given independent data. The objective function takes a tuple of hyperparameters and returns the associated loss.

In such case, the hyperparameter tuning is achieved by using the data obtained at the end of the creating step.

In the specific case described, the parameters tuned among a grid of thirty random combinations were:
mtry=the number of predictors randomly sampled at each split,
min_n=the minimum number of data points in a node required for the node to be split further,
tree_depth=the maximum depth of the tree, and
learn_rate=the rate at which the boosting algorithm adapts from iteration-to-iteration.

Such parameters correspond to the fact the extreme gradient boosting technique proposes to split predictors successively (hence the parameters mtry, min_n and tree_depth which determine the splits that can be done) and to apply a weight to the error of a tree. The idea is to converge towards the optimum by small steps.

During the phase of evaluating the predicting function is evaluated by carrying out at least one evaluation technique.

A first example of evaluation technique is calculating the RMSE between the AUC value predicted by the predicting function and the real AUC value.

A second example of evaluation technique is using a robustness test and/or a durability test.

For instance, artificially created sequential errors on the test data sets are used to assess how performances of the models are sequentially reduced.

A third example of evaluation technique is using random permutations. Such algorithm is used to examine the feature importance to predict the AUC value.

A fourth example of evaluation technique comprises using a bootstrapping technique. Such bootstrapping technique is used to generate confidence intervals on the prediction.

At the end of carrying out all the steps of the assessing method, an AUC value is obtained.

As shown in the experimental section, such method enables to provide with more accurate results with 3 values of concentration.

In some case, satisfactory results are obtained with only 2 values of concentrations.

It should be emphasized that this reduces the number of blood samples to obtain, since there is currently no method, which enables to obtain a good accuracy with less than three blood samples for immunosuppressants.

It is also remarkable that very few parameters are to be provided.

Indeed, one may have imagined that comorbidities and clinical data of the donor would have been crucial data and the Applicant's work shows that it is not the case.

By definition, a comorbidity is the presence of one or more additional conditions co-occurring with a primary condition.

As a specific example, the comorbidity can be whether the donor is living or deceased, if the donor is deceased, whether the cause of the death is due to a circulatory illness, such as a cardiac illness or whether the donor suffers from diabetes. The comorbidities of the subject may also have been considered.

In the described example, there is no need of the age of the donor, the gender of the donor, and the body mass index of the donor. The body mass index is the donor's weight in kilograms divided by the square of height in meters.

Therefore, the assessing method is accurate with very few parameters.

In other words, with such method, the AUC is accessible in a fast and easy way.

In case the system 20 does not have the necessary calculation capabilities for applying the predicting function, calculation can be carried out by interacting with a remote server.

This means that the assessing method is a more convenient and accurate method for measuring AUC.

The present assessing method can be implemented in many different ways. Some examples are given below.

The assessing method may comprise additional step such as outputting the predicted AUC value.

The output can be a graph or an enumeration of values or so on.

Preferably, the output is displayed on the output device 46 of the system 20.

For instance, at the providing step, only specific parameters are provided, such as the concentration values.

As another example, more parameters are provided such as the ethnicity of the subject.

In addition, the assessment method is carried out for another organ.

For instance, instead of considering a graft which is a kidney, the graft is a heart or a lung or a liver.

Such method is, in addition, easy to implement since such method can be carried out by entering subject parameters which are generally known or that can be measured in a non-invasive way. Such entering action as well as carrying out the method can be achieved by using a system 20 which is generally available in each care unit.

In case the system 20 does not have the necessary calculation capabilities for applying the predicting function, calculation can be carried out by interacting with a remote server.

In each of these cases, there is no need of additional hardware resource in the care unit.

Besides, as no invasive act is carried out, the resource allocated to carry out the invasive acts is saved and can be allocated to other tasks.

In addition, such assessing method is appropriate for many different contexts.

Any immunosuppressant can be considered.

Notably, the immunosuppressant can be an inosine mono-phosphate deshydrogenase inhibitor, such as Mycophenolate mofetil, a calcineurin inhibitor: tacrolimus, cyclosporine or a mammalian target of rapamycin (m-TOR) inhibitor, such as everolimus or sirolimus.

Any organ may be considered, such as kidney, heart, lung, liver or hematopoietic stem cell.

In an embodiment, the organ is not a graft. This is notably the case if the subject suffers from autoimmune disorder.

Such advantages of the assessing method renders this method appropriate for many applications.

The assessing method is also advantageous in a method for monitoring patients enrolled in a clinical trial to provide a quantitative measure for the therapeutic efficacy of the therapy which is subject to the clinical trial by carrying out the steps of the assessing method on said patients.

More generally, the assessing method can advantageously be used in any context where the histological piece of information is used and, even more in the case where such histological piece of information can only be obtained in an invasive way.

In addition, the person skilled in the art can consider any combination of the features of the previously mentioned embodiment of the assessing method to obtain new embodiments when the features are technically compatible.

Experimental Section

The efficiency of the assessing method described before is now shown for three different immunosuppressants which are tacrolimus, ciclosporin and mycophenolic acid.

First Immunosuppressant: Tacrolimus

Material and Methods

Patients and Data

Tacrolimus requests from ISBA website were extracted and cleaned using the tidyverse framework. Patient with a renal transplantation and tacrolimus concentrations measured using HPLC methods were selected. The requests including at least 3 sample times at 0 minutes (min), 60 min and 180 min after dose intake were selected with a range of selection of 30 min to 100 min for the 60 min and 140 min to 220 min for the 180 min (the 0, 60, 180 min is the optimal sampling schedule for Prograf® and Advagraf® while the optimal sampling times requested for Envarsus® is 0, 8 and 12 h after dose intake and this later formulation was not included in the analysis).

The dataset was split into two datasets according to the tacrolimus interdose and two independent prediction functions were developed, one for twice a day formulations (TAD; Prograf®) and one for once a day formulations (OAD; Advagraf®).

The other predictors available were the morning dose of tacrolimus, the delay between the request of the graft and the transplantation of the graft and age.

The code used for data cleaning was made using Tidyverse functions such as select, mutate, filter including fct_recode, str_c, dmy or case when.

Plan of the Study

The present study used supervised learning to predict the inter-dose AUC for which the reference value have been obtained by MAP-BE in the ISBA website based on 3 concentrations.

The Applicant developed four predicting functions, which are two predicting functions for both AUC0-12 and AUC-0-24 h predictions (based on 2 and 3 concentrations respectively). The predicting function for AUC0-12 based on 2 concentrations is named F₂^{12h}, the predicting function for AUC0-12 based on 3 concentrations is named F₃^{12h}, the predicting function for AUC0-24 based on 2 concentrations is named F₂^{24h}, the predicting function for AUC0-24 based on 3 concentrations is named F₃^{24h},

A training set was used to build and tune the hyperparameters and evaluate the predicting functions performances by cross-validation.

Once the best predicting function has been defined, it was evaluated on an independent test set that has not been used to develop the predicting function. Such evaluation was carried out by measuring the root mean square error (RMSE) express in μg*h/L between the estimated AUC and reference AUC.

As the reference AUC can be considered as a biased estimators of the “truth” AUC (trapezoidal rule AUC), the Applicant investigated in a second time the performances of the predicting functions developed based on 2 or 3 concentrations on “truth” AUC obtained by trapezoidal rule from MAP-BE in external validation datasets.

For that, two dataset of Prograf® in renal transplant patients, one in liver transplant patients and one in heart transplant patient and one of Advagraf® in renal transplant patients and one in liver transplant patients were used. The performances were also compared to the ones of MAP-BE.

Feature Engineering

The tacrolimus concentrations were binned into 3 theoretical time classes (concentrations at trough (C0 sampled at t=0 min), at 1 h (C1 sampled between 30 and 100 min) and at 3 h (C3 sampled between 140 and 220 min), leading to 3 columns per patient.

To account for the deviation from the theoretical sampling time, new variables were drawn for time 1 and 3 h corresponding to the relative deviation with respect to the theoretical time. To illustrate that, if the sample time was 1.06 h and the corresponding theoretical time was 1 h, the relative time difference was (1.06-1)/1=0.06.

Other predictors were created corresponding to the difference between the concentrations C1-C0, C1-C3 and C3-C0 to add information about the delayed absorption peaks.

Finally, the set of features used to predict interdose AUCs for the predicting functions based on 3 concentrations, namely F₃^{12h} and F₃^{24h}, were:
the delay between the request and transplantation,
age,
morning tacrolimus dose,
tacrolimus concentrations at time 0, 1 h and 3 h after dose intake,
relative deviation from theoretical time and differences between concentrations.

Finally, for the predicting functions, namely F₂^{12h} and F₂^{24h}, based on two concentrations (C0 and C3), the relative time difference for time=1 h and concentrations difference including 01 were removed.

Exploratory Data Analyses

A correlation matrix and scatterplots were drawn to explore the correlation between AUC and predictors using the GGally package.

Pre-Processing of the Data

For all the machine learning analyses, the tidymodels framework was used.

No pre-processing was applied to the data as extreme gradient boosting does not require normalization steps prior to the analysis.

There was no missing data in the predictors.

Data splitting was performed by random selection of patients in a training (75%) and a test set (25%).

Development of the Predicting Functions

The four predicting functions, namely F₂^{12h}, F₂^{24h}, F₃^{12h} and F₃^{24h}, were tuned by searching the parameter combination associated with the lowest RMSE and highest r² with reference AUC values, using a 10-fold cross-validation for which the training dataset was randomly split into 10 parts.

In brief, the best combination of parameters was investigated in 90% of the training dataset (analysis set) and evaluated in the 10% remaining (assessment) dataset, and this process was repeated 10 times.

The parameters tuned among a grid of thirty random combinations were:
the number of predictors randomly sampled at each split (mtry, between 1 and 11),
the minimum number of data points in a node required for the node to be split further (min_n between 1 and 40),
the maximum depth of the tree (tree_depth, between 1 and 15), and
the rate at which the boosting algorithm adapts from iteration-to-iteration (learn_rate, between 0 and 0.08).

Once the best combination of hyperparameters was selected, the relative importance of the predictors was evaluated by random permutations and a variable importance plot was drawn.

Secondly, best parameter combination prediction formulas were evaluated using additional 10-fold cross-validations to assess the mean RMSE and r² and their standard deviations in the train set and scatter plot of estimated AUC as function of reference AUC were drawn.

Finally, AUC predictions were performed using in the test set. The prediction estimation performance were evaluated by RMSE and r² and by calculation of the relative mean prediction error (MPE), the relative RMSE, the number and proportion of estimations with a MPE value out of the ±20% interval. Scatter plots of AUC predicted as function of reference values and residual as function of reference values were drawn.

Evaluation of the Results

Concentrations at 0, 1 and 3 h as well as dose, sampling times and delay between request and transplantation were extracted from the clinical studies database to predict the AUC using the predicting functions and the MAP-BE technique. The full concentrations available in each study were used to calculate the “truth” trapezoidal AUC using the PKNCA package.

Performances of the predicting functions and the MAP-BE technique were compared to the trapezoidal AUC in terms of relative MPE and RMSE, proportion of bias out of the ±10 and ±20% interval.

Additionally, scatter plot of predicted as function of reference AUC and residuals as function of reference AUC were drawn on the same graph for each approach to allow visual comparison.

A linear mixed effect model was built with random effect on “subject” to assess the differences in bias between the predicting functions to estimate AUC (comparison of MPE).

The PCCP study included 137 full pharmacokinetic profiles (ie with numerous available samples) of 11 samples (0, 0.33, 0.66, 1, 1.5, 2, 3, 4, 6, 8, 12 h post dosing) sampled at 7 and 14 days, 1, 3 and 6 months post renal transplantation.

The Prograf and Advagraf patients of the AADAPT study included 34 full PK of 10 samples (0, 0.33, 0.66, 1, 2, 3, 4, 6, 8, 12 h post dosing) and 41 full PK of 13 samples (0, 0.33, 0.66, 1, 2, 3, 4, 6, 8, 12 h, 13 h, 15 h and 24 h post dosing) respectfully collected at 7 days and 3 months post renal transplantation.

The Prograf and Advagraf in liver transplant patients (PALTP study) included 68 full PK of 9 samples (0, 0.5, 1, 2, 3, 4, 6, 8, 12 h post dosing) and 91 full PK of 17 samples (0, 1, 2, 3, 4, 6, 8, 12, 12.5, 13, 14, 15, 16, 18, 20 and 24 h post dosing) respectfully collected at 7 days and 3 months post liver transplantation.

The Pigrec study included 47 full PK of 11 samples (0, 0.33, 0.66, 1, 1.5, 2, 3, 4, 6, 8, 12 h post dosing) sampled at 7 1, 3 and 12 months post cardiac transplantation.

Results

Patients and Data

A total of 4771 and 1449 ISBA requests were available in the cleaned datasets performed in 1912 and 773 patients for each predicting function respectively. Characteristics of the train and test sets are reported in Table 1. The interdose AUCs range between 22 and 380 μg*h/L and between 44 and 698 μg*h/L for TAD and OAD respectively.

TABLE 1
Characteristics of the train and test sets

| | | TAD | | OAD | |
|---|---|---|---|---|---|
| | | Train | Test | Train | Test |
| PARAMETER | VALUE | (n = 3708) | (n = 1236) | (n = 1184) | (n = 394) |
| Time between request | Median | 1.93 | 1.93 | 1.96 | 2.01 |
| and transplantation | Minimum | 0.27 | 0.27 | 0.33 | 0.37 |
| (years) | Maximum | 5.91 | 5.48 | 4.99 | 5.95 |
| Interdose AuC | Median | 128.00 | 130.50 | 236.00 | 222.00 |
| (mg · h/L) | Minimum | 102.00 | 103.00 | 189.00 | 179.25 |
| | Maximum | 162.00 | 164.25 | 292.00 | 275.75 |
| Age (year) | Median | 49.8 | 49.9 | 53.1 | 52.3 |
| | Minimum | 31.0 | 31.6 | 41.4 | 42.3 |
| | Maximum | 61.4 | 61.8 | 62.5 | 62.0 |
| Morning dose (mg) | Median | 2.5 | 2.5 | 6.0 | 5.25 |
| | Minimum | 1.5 | 1.5 | 4.0 | 4.0 |
| | Maximum | 4.0 | 4.0 | 10.0 | 9.0 |
| Through level C0 | Median | 14.70 | 15.40 | 12.60 | 12.31 |
| (μg/L) | Minimum | 10.20 | 10.50 | 9.00 | 8.60 |
| | Maximum | 21.41 | 21.70 | 18.40 | 17.08 |
| Concentration C1 at | Median | 14.70 | 15.40 | 12.60 | 12.31 |
| 1 h (μg/L) | Minimum | 10.20 | 10.50 | 9.00 | 8.60 |
| | Maxmum | 21.41 | 21.70 | 18.40 | 17.08 |
| Concentration C3 at | Median | 12.40 | 12.80 | 13.90 | 13.00 |
| 3 h (μg/L) | Minimum | 9.30 | 9.60 | 9.80 | 9.51 |
| | Maximum | 16.60 | 16.90 | 18.45 | 17.88 |
| Deviation from | Median | 0.00 | 0.00 | 0.00 | 0.00 |
| theoretical time at 1 h | Minimum | 0.00 | 0.00 | 0.00 | 0.00 |
| | Maximum | 0.08 | 0.07 | 0.08 | 0.08 |
| Deviation from | Median | 0.00 | 0.00 | 0.00 | 0.00 |
| theoretical time at 3 h | Minimum | −0.01 | −0.02 | −0.13 | −0.27 |
| | Maximum | 0.02 | 0.02 | 0.01 | 0.01 |
| Difference between | Median | 7.25 | 7.77 | 5.71 | 5.62 |
| C1 and C0 | Minimum | 3.14 | 3.36 | 2.43 | 2.38 |
| | Maximum | 13.19 | 13.16 | 10.93 | 9.76 |
| Difference between | Median | 2.10 | 2.20 | −0.50 | −0.60 |
| C1 and C3 | Minimum | −1.20 | −1.30 | −3.40 | −3.3.5 |
| | Maximum | 7.20 | 7.00 | 2.85 | 2.30 |
| Difference between | Median | 5.22 | 5.41 | 6.84 | 6.61 |
| C3 and C0 | Minimum | 2.92 | 3.16 | 3.70 | 3.57 |
| | Maximum | 8.09 | 8.42 | 10.95 | 10.58 |

| | | | | | | |
|---|---|---|---|---|---|---|
| Type of dose | Advagraf | Not applicable | 1118 | (94.4) | 376 | (95.4) |
| | Envarsus | Not applicable | 66 | (5.6) | 18 | (4.6) |

Exploratory Data Analyses

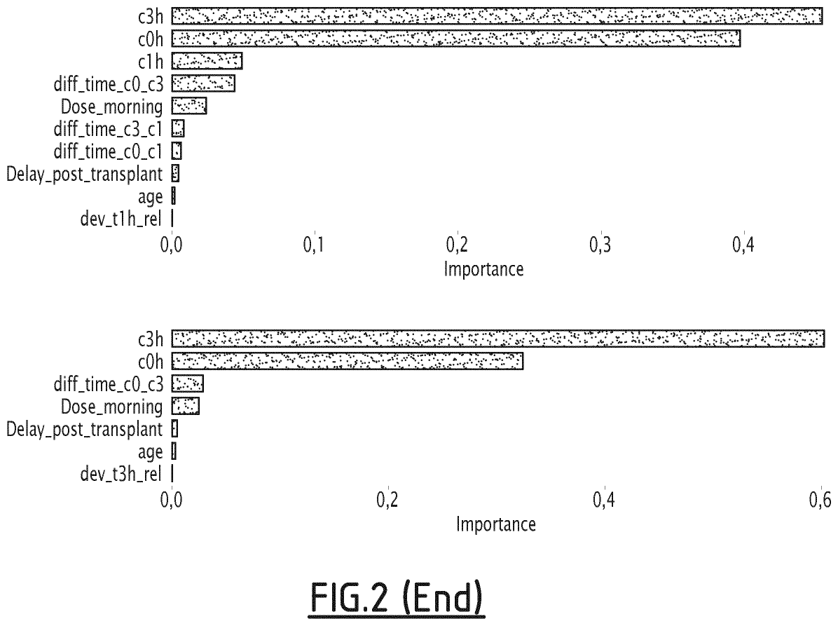

Correlations matrix between AUC and predictors are presented in FIG. 2 showing strong correlation (>0.8) between interdose AUC and C0 or C3h for TAD and C0 for OAD.

In FIG. 2, from above to below, are presented variable importance plot for the predicting functions TAD with 3 concentrations (first graph), with 2 concentrations (second graph) and for the predicting functions OAD with 3 concentrations (third graph), with 2 concentrations (fourth graph).

Predicting Functions for Tad

Predicting Functions F₃^{12h} and F₃^{24h} (with 3 Concentrations)

The performances obtained for the predicting function after 10-fold cross-validation were:
mean±SD RMSE=7.09±0.32 mg*h/L,
r²=0.978±0.002 with a relative MPE=0.2% and RMSE=5.1%.

The best tuned parameters values were:
mtry=9,
min_n=35,
tree_depth=10, and
learning_rate=0.0776.

The variable importance plot of the model is presented in FIG. 2, showing that concentrations at time 3, 0 h post dose and difference between C0 and C3 respectively were the variables of highest importance.

The performances observed in the test set were similar with:
RMSE=7.7 mg*h/L, and
r²=0.977 with a relative MPE=0.3% and RMSE=4.7% and only 9 (0.7%) bias out of the ±20% interval.

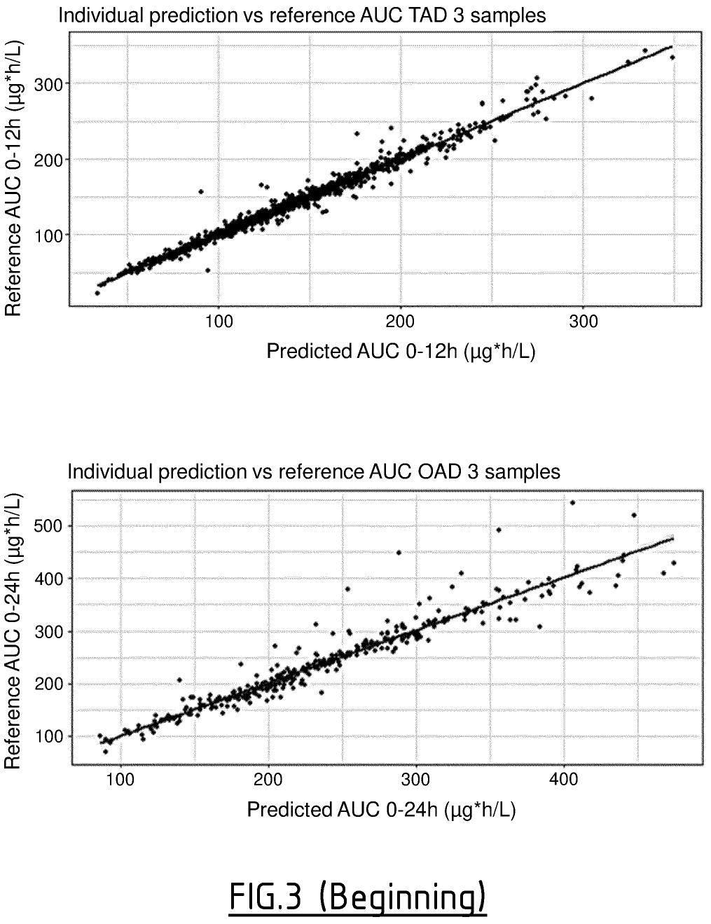

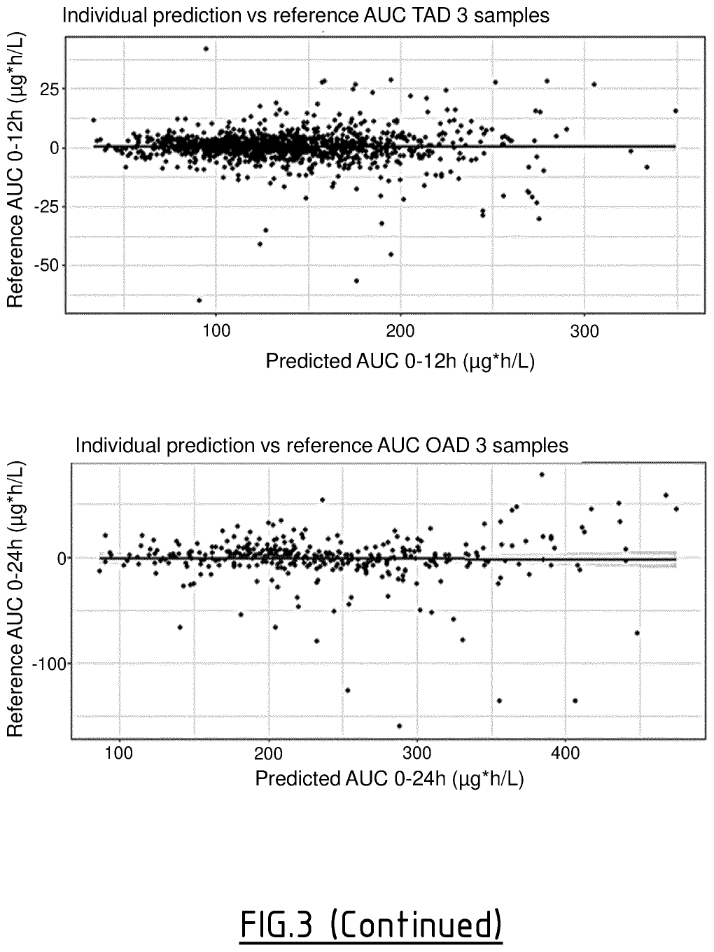

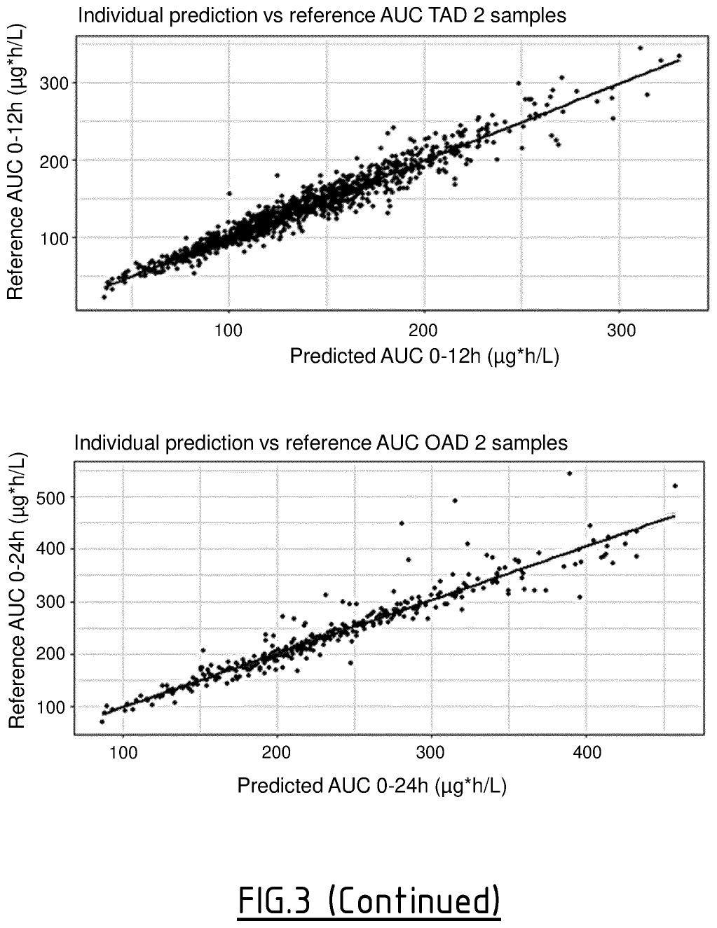

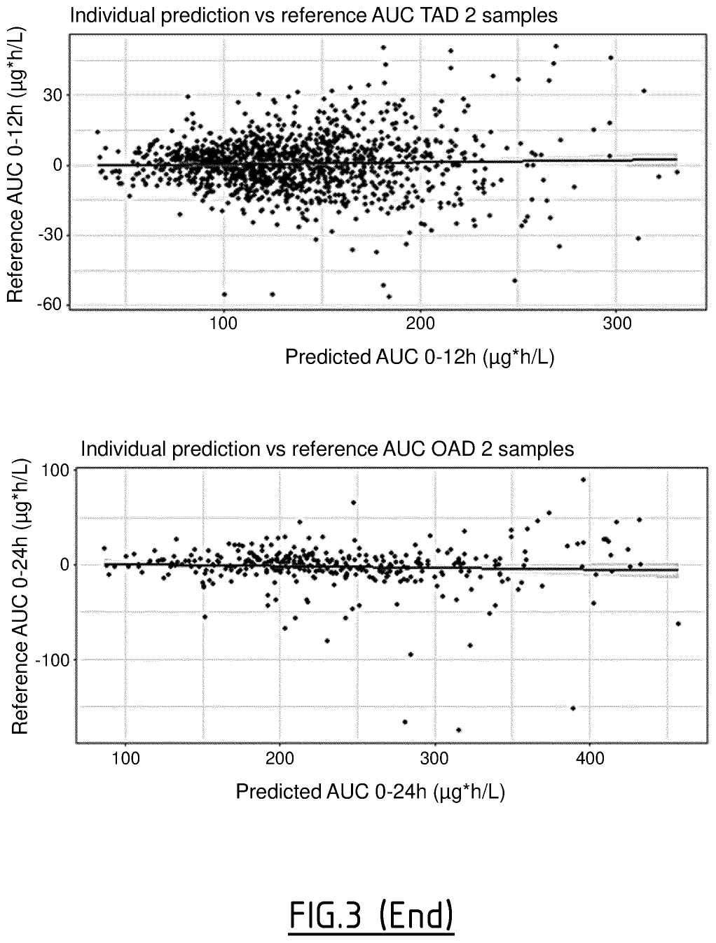

Scatter plot and residual plots are presented in FIG. 3. This figure represents scatter plot and residual plot of reference versus predicted interdose AUC in the test set for predicting functions TAD (first to fourth graphs) and OAD (fifth and eightieth graphs) with 2 or 3 concentrations.

Predicting Functions F₂^{12h} and F₂^{24h} (2 Concentrations)

The performances obtained for the predicting function after 10-fold cross-validation were:
mean±SD RMSE=12.1±0.262 mg*h/L, and
r²=0.936±0.002 with a relative MPE=0.7% and RMSE=8.8%.

The best tuned parameters values were:
mtry=5,
min_n=31,
tree_depth=9, and
learning_rate=0.0126.

The variable importance plot of the model is presented in FIG. 2 showing that concentrations at time 3, 0 h post dose and difference between C0 and C3 respectively were the variables of highest importance.

The performances observed in the test set were similar with:
RMSE=12.2 mg*h/L and
r²=0.941 with a relative MPE=1.0% and RMSE=8.5% and 38 (3.2%) bias out of the ±20% interval.

Corresponding scatter plot and residual plots are presented in FIG. 3 (third and fourth graphs).

Predicting Functions for OAD

Predicting Functions F₃^{12h} and F₂^{12h} (with 3 Concentrations)

The performances obtained for the predicting function after 10-fold cross-validation were:
mean±SD RMSE=20.2±1.25 mg*h/L and
r²=0.938±0.006 with a relative MPE=0.8% and RMSE=8.9%.

The best tuned parameters values were:
mtry=9,
min_n=31,
tree_depth=9, and
learning_rate=0.0126.

The variable importance plot of the model is presented in FIG. 2, showing that concentrations at time 0, 3 and 1 h post dose respectively were the variables of highest importance.

The performances observed in the test set were similar with:
RMSE=22.7 mg*h/L and
r²=0.917 with a relative MPE=0.15% and RMSE=7.7% and 12 (3.3%) bias out of the ±20% interval.

Scatter plot and residual plots are presented in FIG. 3 (fifth and sixth graphs).

Predicting Functions F₂^{12h} and F₂^{24h} (2 Concentrations)

The performances obtained for the predicting function after 10-fold cross-validation were:
mean±SD RMSE=21.3±1.32 mg*h/L and
r²=0.931±0.005 with a relative MPE=0.8% and RMSE=9.2%.

The best tuned parameters values were:
mtry=7,
min_n=31,
tree_depth=9, and
learning_rate=0.0126.

The variable importance plot of the model is presented in FIG. 2, showing that concentrations at time 3, 0 h post dose and difference between C0 and C3 respectively were the variables of highest importance.

The performances observed in the test set were similar with:
RMSE=24.0 mg*h/L and
r²=0.908 with a relative MPE=−0.1% and RMSE=8.2% and 14 (3.9%) bias out of the ±20% interval.

Scatter plot and residual plots are presented in FIG. 3 (seventh and eightieth graphs).

External Evaluation Vs the Trapezoidal Auc and Comparison to POPPK and MAP-BE

The results of the external evaluation are presented in Table 2 and showed that the predicting functions F₂^{12h} and F₂^{24h} with two concentrations led to acceptable results in comparison to two Predicting functions F₃^{12h} and F₃^{24h} with 3 concentrations or MAP-BE technique.

TABLE 2
Comparison of the performances of the predicting
function developed and MAP-technique when the
full profile trapezoidal rule AUC is used

| | | Rela- | Rela- | | |
|---|---|---|---|---|---|
| | | tive | tive | | |
| | | MPE | RMSE | Bias | Bias |
| Study | Method | (%) | (%) | out ±20% n | out ±10% n |
| TAD renal | F₂^{12 h} | −1.2 | 9.4 | 3 | 41 |
| (PCCP) | F₃^{12 h} | 2.5 | 8.2 | 2 | 30 |
| n = 137 | MAP-BE | 5.1 | 9.6 | 6 | 37 |
| TAD renal | F₂^{12 h} | −2.5 | 10.1 | 2 | 9 |
| (AADAPT)) | F₃^{12 h} | −0.4 | 8.4 | 1 | 7 |
| n = 34 | MAP-BE | 2.8 | 9.1 | 1 | 9 |
| OAD renal | F₂^{24 h} | −0.7 | 10.8 | 3 | 15 |
| (AADAPT) | F₃^{24 h} | −1.8 | 11.1 | 3 | 15 |
| n = 41 | MAP-BE | 2.1 | 13.2 | 6 | 16 |
| TAD liver | F₂^{24 h} | 2.4 | 11.1 | 4 | 24 |
| (PALTP) | F₃^{24 h} | 3.3 | 8.7 | 1 | 17 |
| n = 68 | MAP-BE | −3.4 | 11.2 | 6 | 18 |
| OAD liver | F₂^{24 h} | −0.4 | 12.2 | 11 | 32 |
| (PALTP) | F₃^{24 h} | −1.0 | 11.4 | 10 | 33 |
| n = 91 | MAP-BE | −3.5 | 15.7 | 17 | 49 |
| TAD cardiac | F₂^{12 h} | −0.0 | 11.9 | 5 | 18 |
| (PIGREC) | F₃^{12 h} | 4.6 | 13.3 | 9 | 19 |
| n = 47 | MAP-BE | −0.7 | 9.1 | 1 | 13 |

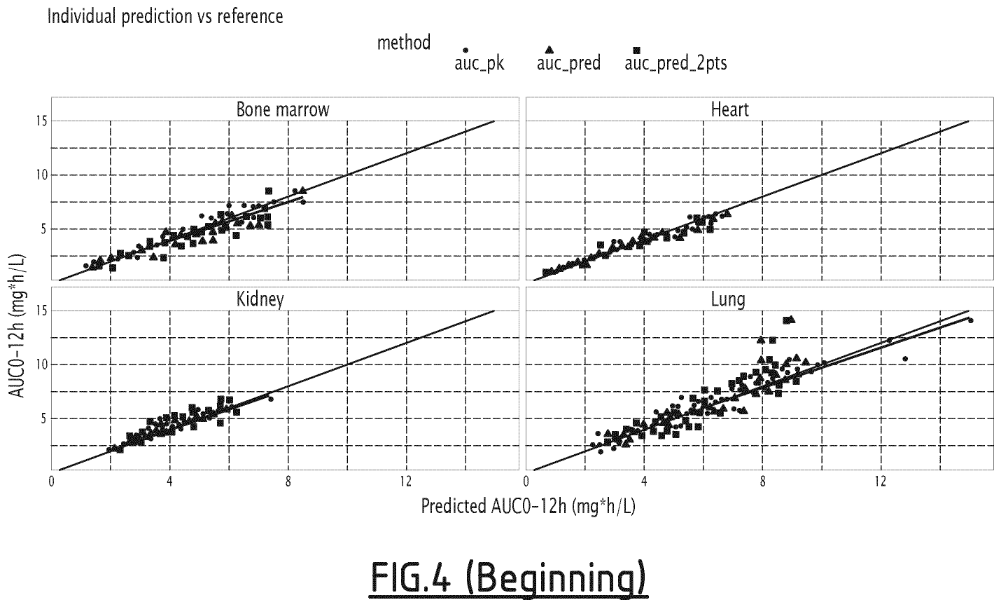

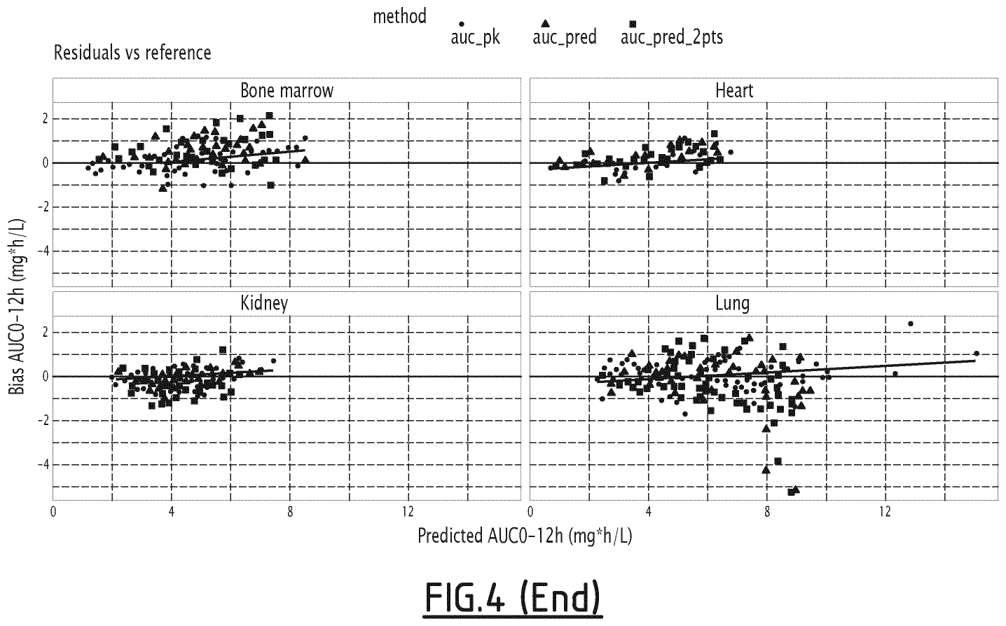

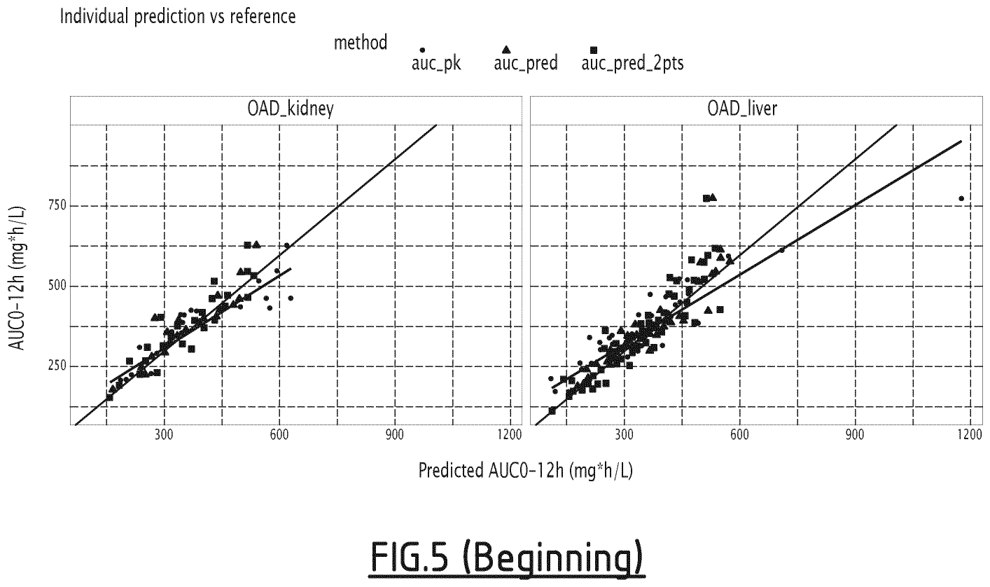

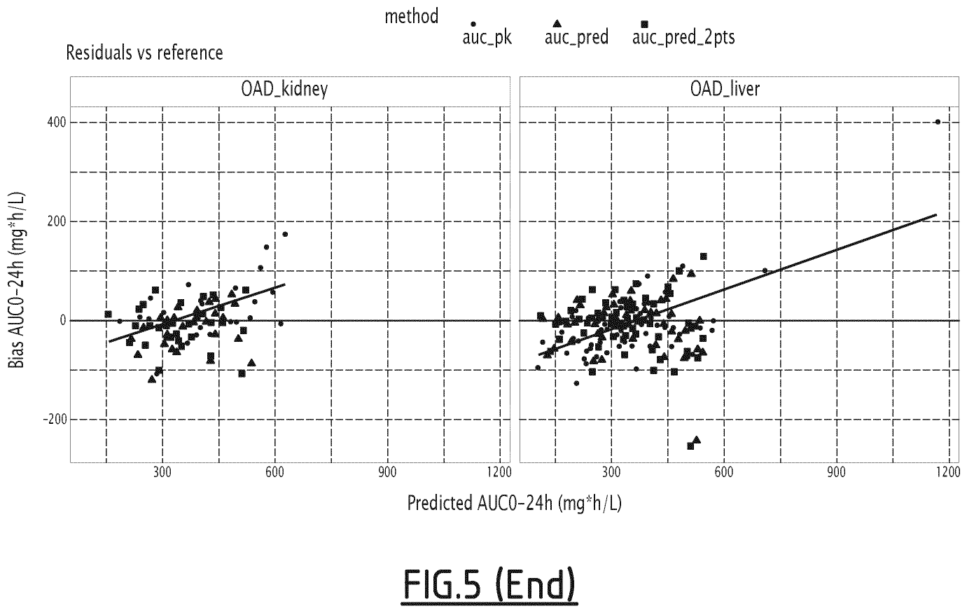

FIGS. 4 and 5 present the scatter plots and residual plots of estimated AUC as function of reference AUC for TAD and OAD respectively in the validation studies.

More precisely, FIG. 4 corresponds to Scatter plot and residual plots of trapezoidal rule AUC vs predicted interdose AUC in the external dataset for predicting functions (auc_pred 2 pts for two concentration values and auc_pred for three concentration values) and for MAP-BE techniques (auc_pk) for TAD heart, renal and liver transplant patients in external full sample validation studies.

Concerning FIG. 5, it represents scatter plot and residual plots of trapezoidal rule AUC vs predicted interdose AUC in the external dataset for predicting functions (auc_pred 2 pts for two concentration values and auc_pred for three concentration values) and for MAP-BE techniques (auc_pk) for OAD renal and liver transplant patients in external full sample validation studies.

The predictions obtained in the current work are excellent in comparison to POPPK results usually obtained with 71% showing bias of 10% or less but only 39% showing an imprecision of 10% or less for the Bayesian forecasting strategies that used two or more tacrolimus concentrations. Using predicting functions, the Applicant found RMSE around or less than 10% and bias lower than 5% and a very low number of profile out of the ±20% relative bias interval in external dataset.

Second Immunosuppressant: Ciclosporin

Material and Methods

A experiment similar to the one carried out for tacrolimus was carried out by the Applicant for ciclosporin.

Therefore, many common elements are shared between the two experiments and are not repeated hereinafter, so that only the differences other that replacing tacrolimus by cyclosporin are underlined.

For patients and data, patient with a renal, heart, liver, lung, bone marrow transplantations and nephrotic syndrome or auto-immune diseases gathered in “other” categories were extracted with their respective ciclosporine concentrations measured using HPLC or EMIT methods.

In addition, only one predicting function per number of concentrations (respectively 2 and 3) is calculated.

Datasets of heart transplant, one of bone marrow transplant, 1 of lung, 3 of renal measured using LCMS, 1 of renal measured using EMIT, 1 of renal measured using FPIA were used.

In the development, the number of predictors randomly sampled at each split, mtry, was comprised between 1 and 21 instead of between 1 and 11.

The first kidney study (Debord et al clinpk 2001) included 21 full PK of 10 samples (0, 0.66, 1, 1.5, 2, 3, 4, 6, 8 h post dosing) in stable renal transplant recipients. The second kidney study is the stablocine with sample measured using LCMS, EMIT and FPIA for 20 full PK of 10 samples (0, 0.33, 0.66, 1, 1.5, 2, 3, 4, 6, 9 h post dosing) in stable renal transplant recipients. The third kidney study is the concept study with sample measured using LCMS for 20 full PK of 11 samples (0, 0.33, 0.66, 1, 1.5, 2, 3, 4, 6, 8 h and 12 h post dosing) in stable renal transplant recipients. The lung transplants of the stimmugrep study included 79 full PK of 11 samples (0, 0.33, 0.66, 1, 1.5, 2, 3, 4, 6, 8, 12 h post dosing) sampled at 7 days, 1, 3 and 12 months post cardiac transplantation in 37 patients. The Pigrec study included 33 full PK of 11 samples (0, 0.33, 0.66, 1, 1.5, 2, 3, 4, 6, 8, 12 h post dosing) sampled at 7 days, 1, 3 and 12 months post cardiac transplantation in 11 patients. Bone marrow transplant patients from a study performed in Limoges University Hospital consisted in 72 rich PK profiles (0, 0.33, 0.66, 1, 2, 3, 4, 6, 8, 12 h post dosing) sampled in 45 patients.

Results

Patients and Data

A total of 6360 ISBA requests were available in the cleaned datasets performed in 2009 patients. Characteristics of the train and test sets are reported in Table 3. The interdose AUCs range between 0.28 and 10.40 mg*h/L.

Table 1: Characteristics of the ISBA requests used for the development and validation of the models

TABLE 3
Characteristics of the train and test sets

| | | Train | | Test | |
|---|---|---|---|---|---|
| PARAMETER | VALUE | (n = 4770) | | (n = 1590) | |
| Time between request | Median | 7.75 | | 7.99 | |
| and transplantation | Minimum | 1.28 | | 1.89 | |
| (years) | Maximum | 14.00 | | 14.38 | |
| Interdose AUC | Median | 2.79 | | 2.76 | |
| (mg*h/L) | Minimum | 2.31 | | 2.29 | |
| | Maximum | 3.58 | | 3.57 | |
| age (year) | Median | 59.59 | | 59.73 | |
| | Minimum | 48.06 | | 48.39 | |
| | Maximum | 67.65 | | 67.97 | |
| Morning dose (mg) | Median | 85.00 | | 80 | |
| | Minimum | 70.00 | | 70 | |
| | Maximum | 110.00 | | 100 | |
| Trough level (C0) | Median | 94.00 | | 93.00 | |
| (μg/L) | Minimum | 74.00 | | 73.00 | |
| | Maximum | 125.00 | | 125.00 | |
| Concentration at 1 h; | Median | 676.00 | | 680.00 | |
| C1 (μg/L) | Minimum | 475.00 | | 464.00, | |
| | Maximum | 890.00 | | 904.75 | |
| Concentration at 3 h; | Median | 324.00 | | 318.50 | |
| C3 (μg/L) | Minimum | 259.00 | | 254.00 | |
| | Maximum | 428.00 | | 428.00 | |
| Deviation from | Median | 0.00 | | 0.00 | |
| theoretical time at 1 h | Minimum | 0.00 | | 0.00 | |
| | Maximum | 0.03 | | 0.03 | |
| Deviation from | Median | 0.00 | | 0.00 | |
| theoretical time at 3 h | Minimum | 0.00 | | 0.00 | |
| | Maximum | 0.03 | | 0.03 | |
| Differences between | Median | 574.00 | | 573.00 | |
| C1 and C0 | Minimum | 381.00 | | 369.00 | |
| | Maximum | 780.00 | | 797.00 | |
| Differences between | Median | 349.50 | | 344.00 | |
| C1 and C3 | Minimum | 143.00 | | 136.00, | |
| | Maximum | 535.00 | | 544.75 | |
| Differences between | Median | 228.00 | | 225.00 | |
| C3 and C0 | Minimum | 175.00 | | 172.00 | |
| | Maximum | 304.00 | | 306.00 | |
| Indication | Other | 13 | (0.3) | 7 | (0.4) |
| | Heart trans- | 12 | (0.3) | 4 | (0.3) |
| | plantation | | | | |
| | Liver | 16 | (0.3) | 12 | (0.8) |
| | Bone Marrow | 286 | (6.0) | 100 | (6.3) |
| | Lung | 7 | (0.1) | 2 | (0.1) |
| | Kidney | 4418 | (92.6) | 1462 | (91.9) |
| | Nephrotic | 18 | (0.4) | 3 | (0.2) |
| | syndrom | | | | |

Exploratory Data Analyses

As for tacrolimus, correlations matrix between AUC and predictors show strong correlation (>0.8) between interdose AUC and C0 or C3h.

Predicting Functions

Predicting Function with 3 Concentrations

The performances obtained for the predicting function after 10-fold cross-validation were:
mean±SD RMSE=0.168±0.009 mg*h/L,
r²=0.981±0.002 with a relative MPE=0.28% and RMSE=4.7%.

The best tuned parameters values were:
mtry=12,
min_n=31,
tree_depth=9, and
learning_rate=0.0126.

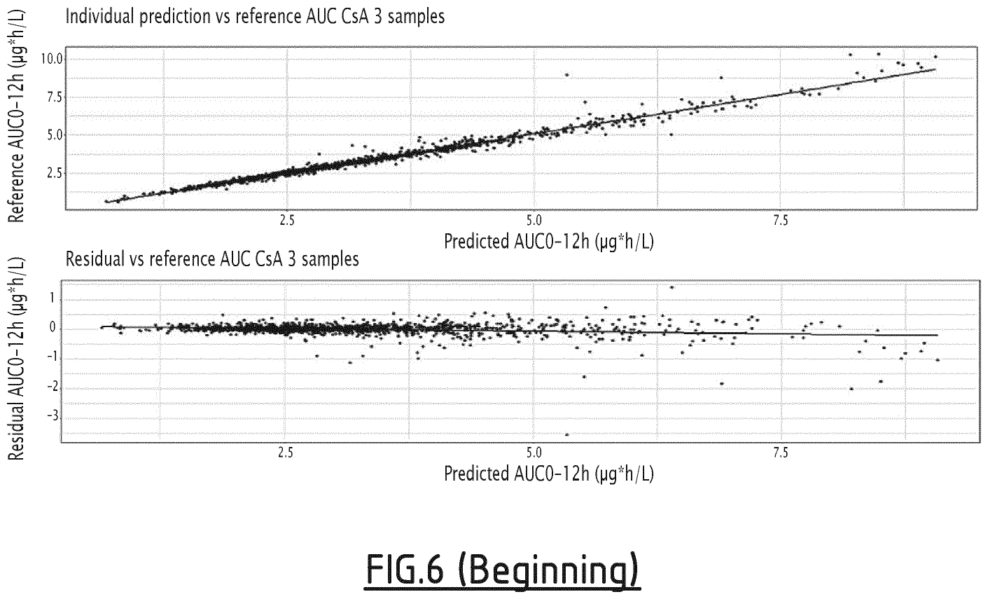

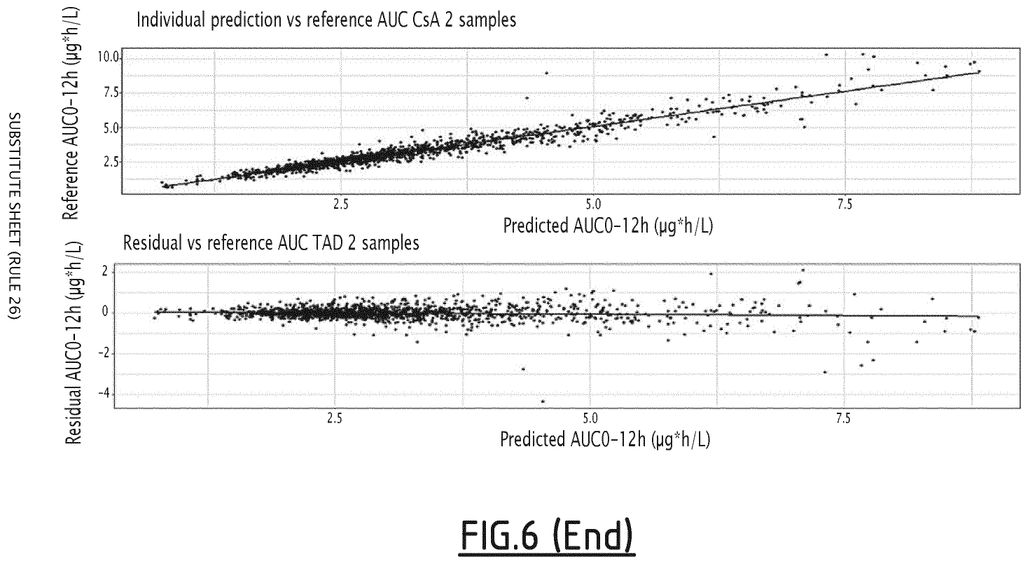

The variable importance plot of the model is presented in FIG. 6, showing that concentrations at time 3, 0 h post dose and difference between C0 and C3 respectively were the variables of highest importance.

The performances observed in the test set were similar with:
RMSE=0.199 mg*h/L, and
r²=0.978 with a relative MPE=0.15% and RMSE=4.35% and only 10 (0.8%) bias out of the ±20% interval.

Predicting Function with 2 Concentrations

The performances obtained for the predicting function after 10-fold cross-validation were:

mean±SD RMSE=0.309±0.008 mg*h/L, and
r²=0.937±0.003 with a relative MPE=0.9% and RMSE=9.8%.

The best tuned parameters values were:
mtry=9,
min_n=31,
tree_depth=9, and
learning_rate=0.0126.

The variable importance plot of the model is presented in FIG. 6 showing that concentrations at time 3, 0 h post dose and difference between C0 and C3 respectively were the variables of highest importance.

The performances observed in the test set were similar with:
RMSE=0.345 mg*h/L and
r²=0.931 with a relative MPE=0.5% and RMSE=9.5% and 85 (5.3%) bias out of the ±20% interval.

External Evaluation Vs the Trapezoidal Auc and Comparison to POPPK and MAP-BE

The results of the external evaluation are presented in Table 4 and showed that the predicting function with 2 concentrations led to acceptable results in comparison to the predicting function with 3 concentrations or the MAP-BE technique specifically in the case of renal and heart transplantations and for EMIT and LCMS dose measurements methods.

TABLE 4
Comparison of the performances of the predicting
function developed and MAP-technique when the
full profile trapezoidal rule AUC is used

| | | Rela- | Rela- | | |
|---|---|---|---|---|---|
| | | tive | tive | Bias | Bias |
| | | MPE | RMSE | out ±20% | out ±10% |
| Study | Method | (%) | (%) | n | n |
| Renal 1 | F2 | −1.7 | 8.8 | 1 | 4 |
| Debord 2001 | F3 | 0.7 | 3.4 | 0 | 0 |
| n = 21 | MAP-BE | 12.3 | 13.6 | 1 | 14 |
| Renal 2 | F2 | −6.9 | 11.0 | 1 | 7 |
| Stablocine LCMS | F3 | −0.2 | 3.8 | 0 | 0 |
| n = 20 | MAP-BE | 10.4 | 12.3 | 1 | 13 |
| Renal 2 | F2 | −10.2 | 11.6 | 1 | 10 |
| Stablocine EMIT | F3 | −1.1 | 3.9 | 0 | 0 |
| n = 20 | MAP-BE | 3.2 | 6.7 | 0 | 3 |
| Renal 2 | F2 | −7.7 | 12.2 | 1 | 10 |
| Stablocine FPIA | F3 | −0.4 | 6.3 | 0 | 3 |
| n = 20 | MAP-BE | −3.2 | 7.5 | 0 | 5 |
| Renal 3 | F2 | −9.9 | 14.5 | 3 | 12 |
| CONCEPT | F3 | −0.0 | 6.3 | 0 | 3 |
| n = 20 | MAP-BE | 7.9 | 11.9 | 2 | 11 |
| TAD cardiac | F2 | 4.8 | 13.8 | 6 | 14 |
| (PIGREC) | F3 | 3.2 | 9.9 | 2 | 8 |
| n = 47 | MAP-BE | −1.1 | 10.1 | 2 | 9 |
| Lung | F2 | 1.4 | 17.2 | 16 | 43 |
| stimmugrep | F3 | 0.9 | 12.9 | 8 | 32 |
| n = 79 | MAP-BE | 0.8 | 10.8 | 6 | 22 |
| Bone marrow | F2 | 9.6 | 18.7 | 13 | 28 |
| n = 72 | F3 | 8.5 | 16.5 | 13 | 29 |
| | MAP-BE | 1.1 | 13.0 | 8 | 24 |

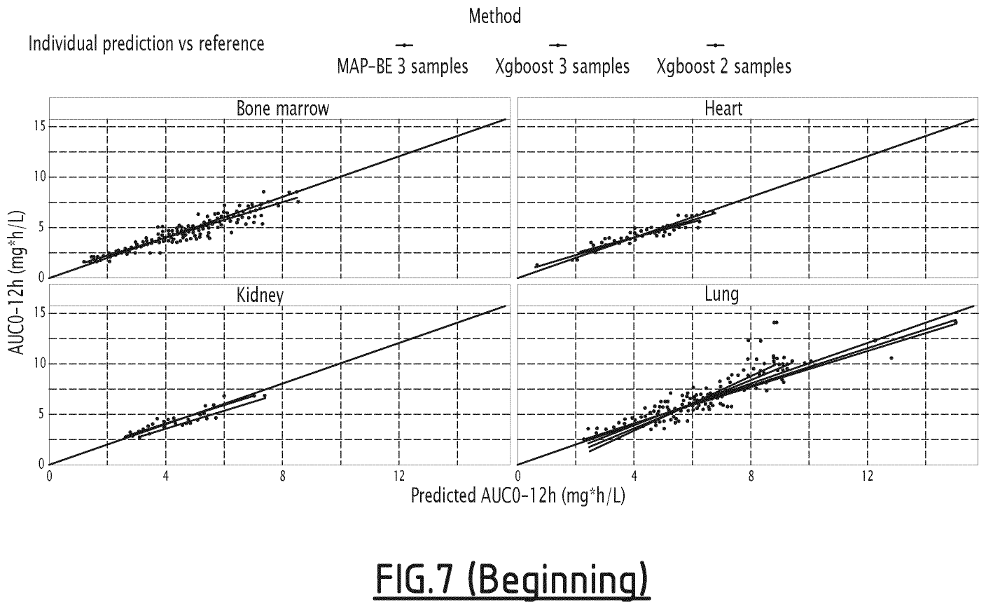

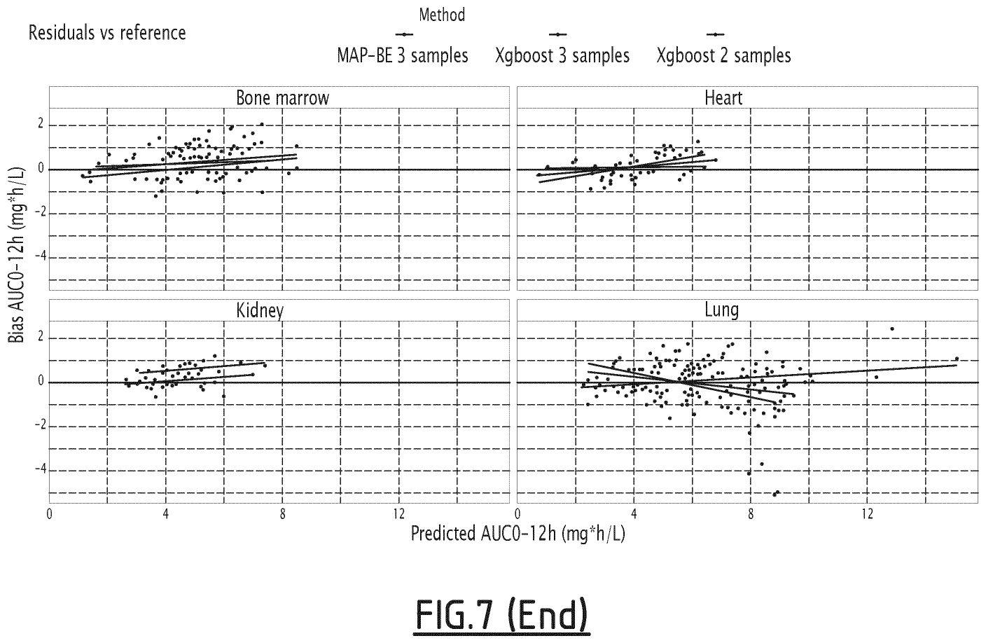

FIG. 7 presents the scatter plots and residual plots of estimated AUC for 2 or 3 concentrations Xgboost models or MAP-BE as function of reference AUC in validation studies.

More precisely, FIG. 7 corresponds to scatter plot and residual plots of trapezoidal rule AUC vs predicted interdose AUC in the external dataset for predicting functions (auc_pred 2 pts for two concentration values and auc_pred for three concentration values) and for MAP-BE techniques (auc_pk) for cyclosporine in heart, renal and liver transplant patients in external full sample validation.

There again, the predictions obtained in the current work are also satisfactory in comparison to POPPK results.

Third Immunosuppressant: Mycophenolic Acid (MPA)

Material and Methods

A experiment similar to the one carried out for tacrolimus was carried out by the Applicant for mycophenolic acid.

Therefore, many common elements are shared between the two experiments and are not repeated hereinafter, so that only the differences other that replacing tacrolimus by mycopheolic acid are underlined.

For patients and data, the dataset was sequentially refined by selecting the requests where MPA plasma levels were measured using HPLC, including at least 3 times of sampling approximately 20 min (10 to 30 min, C20), 60 min (30 to 100 min, C1) and 180 min (140 to 220 min, C3) after dose intake. Actually, the 20, 60, 180 min is the optimal sampling schedule for MMF. A filter was applied to select the requests with an interdose of 12 h. The other predictors available were the morning dose of MMF, the time elapsed between transplantation and MPA plasma sampling, the type of transplant, the associated immunosuppressant and patient age.

For that, 3 datasets in adult renal transplant patients: one dataset of MPA+tacrolimus (PCCP), one of MPA+cyclosporine (Stablocine) and 2 datasets of MPA+cyclosporine or sirolimus (CONCEPT, SPIESSER); one dataset MPA in pediatric renal transplant patients; one dataset in heart transplant patient of MPA+cyclosporine or tacrolimus (Pigrec) were used. The performance of the prediction functions in these confirmation datasets was also compared to that of the MAP-BE technique. On the contrary to calcineurin inhibitors, the reference AUC was not the trapezoidal rule but the AUC obtained by MAP-BE from all the available samples.

Other predictors corresponding to the differences between the concentrations C1-C20, C1-C3 and C3-C20 were created and to add information about potentially delayed absorption peaks, 3 dummy variables corresponding to C3>C1, C3>C20 and 03>C20&C1 were created. The type of transplant was split into 9 categories: kidney, heart, lung, liver, bone marrow, lupus, pediatric lupus, nephrotic syndrome plus an “other” category for all the remaining indications (including transplantation of 2 solid organs or auto-immune diseases).

Finally, the set of features used to predict interdose AUCs were:
the type of transplant,
the associated immunosuppressant (tacrolimus, sirolimus, cyclosporine, or nothing),
patient age,
time elapsed between transplantation and MPA plasma sampling,
MMF morning dose,
MPA concentrations at times 20 min, 1 h and 3 h,
relative deviation from the theoretical times,
differences between concentrations and
dummy variables for delayed absorption peak: when the MPA concentration at 3 h was higher to concentration at 1 h, concentration at 20 min or both, then delayed absorption=1 otherwise=0.

For the predicting function based on 2 concentrations (C1 and C3), the relative time difference for time=20 m and concentration differences or dummy delayed absorption including C20 were removed.

For the development of the predicting functions, a 40×40 grid was used. The number of predictors randomly sampled at each split, mtry, was between 1 and 28 and the amount of data exposed to the fitting routine, sample size, was between 10 and 100%.

For the external evaluation, concentrations at 20 min, 1 h and 3 h as well as dose, sampling times and time elapsed between transplantation and MPA plasma sampling were extracted from the PK databases to predict the AUC using the predicting functions and the MAP-BE technique.

The PCCP study included 128 PK profiles of 11 samples (0, 0.33, 0.66, 1, 1.5, 2, 3, 4, 6, 8, 12 h post dosing) collected at 7 and 14 days, 1, 3 and 6 months after renal transplantation. The stablocine study included 20 PK profiles of 10 or 11 samples (0, 0.33, 1, 1.5, 2, 3, 4, 6, 9 h and (not present in every profiles) 12 h post dosing) in stable renal transplant patients. The CONCEPT study included 67 PK profiles of 10 or 11 samples (0, 0.66, 1, 1.5, 2, 3, 4, 6, 9 h and (not present in every profiles) 12 h post dosing) collected at week 12, 16 and 26 after renal transplantation. The PIGREC study included 75 PK profiles of 10 samples (0, 0.33, 0.66, 1, 1.5, 2, 3, 4, 6 and 9 h post dosing) collected between day 7 and day 14, and at 1, 3 and 12 months after transplantation.

Results

Patients and Data

A total of 12877 mycophenolic acid AUC0-12 h from 6884 patients were available in the cleaned datasets extracted from ISBA. Characteristics of the train and test sets are reported in Table 5.

TABLE 5
Characteristics of the train and test sets

| | | TRAIN | | TEST | |
|---|---|---|---|---|---|
| PARAMETER | VALUE | (N = 9658) | | (N = 3219) | |
| Time between request | Median | 1.07 | | 1.06 | |
| and MPA plasma | Minimum | 0.01 | | 0.01 | |
| concentrations (years) | Maximum | 32.99 | | 29.01 | |
| Interdose AUC0_12 h | Median | 39.30 | | 39.24 | |
| (mg*h/L) | Minimum | 1.12 | | 3.19 | |
| | Maximum | 181.39 | | 163.62 | |
| age (year) | Median | 53.74 | | 52.85 [ | |
| | Minimum | 0.14 | | 0.25 | |
| | Maximum | 94.83 | | 88.42 | |
| Morning dose (mg) | Median | 750.00 | | 750.00 | |
| | Minimum | 80.00 | | 100.00 | |
| | Maximum | 2000.00 | | 2000.00 | |
| Deviation from the 20 | Median | 0.00 | | 0.00 | |
| m theoretical time | Minimum | −0.50 | | −0.50 | |
| | Maximum | 0.50 | | 0.50 | |
| Deviation from the 1 | Median | 0.00 | | 0.00 | |
| h theoretical time | Minimum | −0.25 | | −0.25 | |
| | Maximum | 0.25 | | 0.25 | |
| Deviation from the 3 | Median | 0.00 | | 0.00 | |
| h theoretical time | Minimum | −0.11 | | −0.11 | |
| | Maximum | 0.11 | | 0.11 | |
| Concentration difference | Median | 1.70 | | 1.70 | |
| between C1 h and C20 m | Minimum | −72.00 | | −45.60 | |
| | Maximum | 53.72 | | 48.26 | |
| Concentration difference | Median | 5.20 | | 5.39 | |
| between C1 h and C3 h | Minimum | −31.80 | | −24.80 | |
| | Maximum | 55.26 | | 48.03 | |
| Concentration difference | Median | 2.10 | | 2.30 | |
| between C20 m and C3 h | Minimum | −31.20 | | −24.00 | |
| | Maximum | 82.10 | | 57.20 | |
| Indication | Kidney | 7398 | (76.6) | 2470 | (76.7) |
| | Heart | 782 | (8.1) | 246 | (7.6) |
| | Nephrotic | 524 | (5.4) | 187 | (5.8) |
| | syndrome | | | | |
| | Liver | 296 | (3.1) | 100 | (3.1) |
| | Lung | 202 | (2.1) | 79 | (2.5) |
| | Lupus | 97 | (1.0) | 30 | (0.9) |
| | Pediatric | 45 | (0.5) | 9 | (0.3) |
| | Lupus | | | | |
| | Bone | 34 | (0.4) | 13 | (0.4) |
| | Marrow | | | | |
| | Other | 280 | (2.9) | 85 | (2.6) |

Exploratory Data Analyses

Hereagain, correlation matrices between AUC and predictors shows a strong correlation between interdose AUC and C3h (r=0.7) and C1h (r=0.66).

Predicting Function

The best-tuned parameters values for each model are presented Table 6.

TABLE 6
Optimized parameter values for each predicting model

| Data set | Parameters | F2 | F3 |
|---|---|---|---|
| training | *RMSE ± SD (mg*h/L) | 6.78 ± 0.08 | 5.59 ± 0.07 |
| | *R² ± SD | 0.864 ± 0.004 | 0.908 ± 0.002 |
| | Relative MPE (%) | 2.7 | 1.8 |
| | Relative RMSE (%) | 18.6 | 14.5 |
| testing | RMSE (μg*h/L) | 6.41 | 5.34 |
| | R² | 0.876 | 0.914 |
| | Relative MPE (%) | 2.1 | 1.4 |
| | Relative RMSE (%) | 17.0 | 13.2 |
| | Number of MPE out of | 560 (17.4%) | 306 (9.5%) |
| | the ±20% interval n(%) | | |

*Values and standard deviations obtained after 10 fold crossvalidation,

Results in the training set obtained after 10-fold cross-validation and in the testing set are presented in Table 6 and show excellent results and no difference between them (no overfitting) but with approximately a twice better estimation for 3 concentrations vs 2 concentrations. The relative MPE were close to 0 and the relative RMSE were <20% in both the training and testing sets.

In addition, the variable importance plot of each model shows that concentrations at time 3 and 0 are the most important variables.

External Evaluation

The results of the external evaluation are presented in Table 7 hereinafter and show that the predicting function with 2 concentrations led to acceptable results in comparison to the predicting function with 3 concentrations or the MAP-BE technique.

TABLE 7
Comparison of the performances of the predicting
function developed and MAP-BE technique when the
full profile MAP-BE AUC is used as a reference.

| | | Rela- | Rela- | | | | |
|---|---|---|---|---|---|---|---|
| | | tive | tive | Bias | | Bias | |
| | | MPE | RMSE | out ±20% | | out ±10% | |
| Study | Method | (%) | (%) | n (%) | | n (%) | |
| PCCP study | F2 | −1.2 | 24.2 | 46 | (35.9) | 80 | (62.5) |
| (n = 128) | F3 | −4.0 | 19.0 | 39 | (30.5) | 76 | (59.4) |
| | MAP-BE | 1.0 | 19.9 | 37 | (28.9) | 81 | (63.3) |
| Stablocine | F2 | 1.5 | 17.8 | 4 | (20) | 10 | (50) |
| (n = 20) | F3 | 2.8 | 14.7 | 2 | (10) | 8 | (40) |
| | MAP-BE | 9.3 | 20.5 | 7 | (35) | 9 | (45) |
| CONCEPT | F2 | −0.8 | 26.3 | 27 | (40 | 42 | (63) |
| (n = 67) | F3 | −1.1 | 22.5 | 14 | (21) | 43 | (64) |
| | MAP-BE | 2.1 | 25.1 | 18 | (27) | 40 | (60) |
| PIGREC | F2 | 2.5 | 23.1 | 31 | (41) | 44 | (59) |
| (n = 75) | F3 | −1.2 | 15.6 | 17 | (23) | 41 | (55) |
| | MAP-BE | 3.5 | 17.3 | 17 | (23) | 47 | (63) |

Comparison of the Performances of Several Algorithms

In this paragraph, the question to address is whether the algorithms built using reference AUC estimated from at least 8 samples (using the trapezoidal rule) are better than the ones built using a 3 samples pharmacokinetic-model-based estimated AUC(=abbreviated AUC).

Theoretically, using more precise AUC based on several samples using the trapezoidal rule would improve the performances by decreasing the random noise linked to a model-based estimation of the reference abbreviated AUC. However, this is also associated with a decrease in the sample size available for the development of the algorithms.

The Applicant showed that the performances of the algorithms built using reference AUC for tacrolimus did not show better performances in comparison to the ones previously developed from abbreviated AUC.

TABLE 8
Comparative performance for AUC estimation in an external set
of the final MARS algorithm based on 2 concentrations (0 and
12 h) (best for 2 concentrations), 3 concentrations with MARS
algorithm (0, 8 and 12 h) (best for 3 concentrations), MAP-BE
previously developed based on 3 samples (0, 8, 12 hours; Woillard
et al Clinical Pharmacokinet 2018) and MAP-BE on 3 samples
(0, 4, 8 hours; Moes et al BJCP 2021).

| | Relative | Relative | Relative errors | | Relative errors | |
|---|---|---|---|---|---|---|
| | MPE | RMSE | out of ±20% n | | out of ±10% n | |
| | (%) | (%) | (%) | | (%) | |
| MARS 2 samples | −3.1 | 11.1 | 2 | (12.5%) | 5 | (31.3%) |
| (0 and 12 hours) | | | | | | |
| MARS 3 samples | −4.2 | 10.1 | 1 | (6.3%) | 5 | (31.3%) |
| (0, 8 and 12 hours) | | | | | | |
| MAP-BE 3 samples | 7.3 | 23.0 | 8 | (53.3) | 12 | (80) |
| (0, 8 and 12 hours) | | | | | | |
| MAP-BE 3 samples | −7.4 | 11.2 | 1 | (6.3%) | 7 | (43.8%) |
| (0, 4, 8 hours) | | | | | | |

MARS is multivariate adaptive regression splines

TABLE 9
Performances of ML models and MAP-BE based on 3 samples LSS to estimate
reference AUCs obtained from full PK profiles using the trapezoidal rule.

| | | | | Bias out | | Bias out | |
|---|---|---|---|---|---|---|---|
| | | Relative | Relative | of ±20% | | of ±10% | |
| Study | Method | MPE (%) | RMSE (%) | n (%) | | n (%) | |
| TAC BID | Xgboost | −0.6 | 9.0 | 3 | (2.2) | 42 | (30.6) |
| kidney 1 | 2 concentrations | | | | | | |
| (n = 137) | Xgboost | 2.9 | 8.1 | 2 | (1.4) | 26 | (19.0) |
| | 3 concentrations | | | | | | |
| | MAP-BE | 5.1 | 9.6 | 6 | (4.4) | 37 | (27.0) |
| | 3 concentrations | | | | | | |
| TAC BID | Xgboost | −1.0 | 9.1 | 2 | (5.9) | 9 | (26.5) |
| kidney 2 | 2 concentrations | | | | | | |
| (n = 34) | Xgboost | −0.1 | 7.3 | 0 | (0) | 7 | (20.6) |
| | 3 concentrations | | | | | | |
| | MAP-BE | 2.8 | 9.1 | 1 | (2.9) | 9 | (26.5) |
| | 3 concentrations | | | | | | |
| TAC QD | Xgboost | −0.6 | 10.8 | 3 | (7.3) | 14 | (34.1) |
| kidney | 2 concentrations | | | | | | |
| (n = 41) | Xgboost | −2.0 | 11.1 | 3 | (7.3) | 14 | (34.1) |
| | 3 concentrations | | | | | | |
| | MAP-BE | 2.1 | 13.2 | 6 | (14.6) | 16 | (39.0) |
| | 3 concentrations | | | | | | |
| TAC BID | Xgboost | 3.3 | 12.9 | 7 | (10.3) | 30 | (44.1) |
| liver | 2 concentrations | | | | | | |
| (n = 68) | Xgboost | 4.1 | 9.9 | 3 | (4.4) | 14 | (20.6) |
| | 3 concentrations | | | | | | |
| | MAP-BE | −3.4 | 11.2 | 6 | (8.8) | 18 | (26.5) |
| | 3 concentrations | | | | | | |
| TAC QD | Xgboost | −0.4 | 12.3 | 10 | (11.0) | 35 | (38.5) |
| liver | 2 concentrations | | | | | | |
| (n = 91) | Xgboost | −0.9 | 11.5 | 10 | (11.0) | 31 | (34.1) |
| | 3 concentrations | | | | | | |
| | MAP-BE | −3.5 | 15.7 | 17 | (18.7) | 49 | (53.8) |
| | 3 concentrations | | | | | | |
| TAC BID | Xgboost | −0.4 | 9.7 | 2 | (4.2) | 13 | (27.7) |
| heart | 2 concentrations | | | | | | |
| (n = 47) | Xgboost | 5.4 | 10.8 | 3 | (6.4) | 18 | (38.3) |
| | 3 concentrations | | | | | | |
| | MAP-BE | −0.7 | 9.1 | 1 | (2.1) | 13 | (27.7) |
| | 3 concentrations | | | | | | |

As observed in the tables 8 and 9 above, the performances on external full PK profiles datasets are approximately the same in terms of bias and RMSE.

### Glossary

AADAPT study: see study of the Marquet, P. et al. Pharmacokinetic therapeutic drug monitoring of Advagraf® in more than 500 adult renal transplant patients, using an expert system online. Ther Drug Monit(2018).doi:10.1097/FTD.0000000000000503 Advagraf®: brand name of a drug (commercialized by Astellas Pharma) comprising tacrolimus and given once daily (OAD).
AUC curve: area-under-the-concentration-over-time curve
AUC0-12: AUC between t=0 (time of administration) and t=12 h
AUC0-24h: AUC between t=0 (time of administration) and t=24 h
Debord et al clinpk 2001: it refers to the study that can be found in Debord J, Risco E, Harel M, Le Meur Y, Buchler M, Lachatre G, Le Guellec C, Marquet P. Application of a gamma model of absorption to oral cyclosporin. Clin Pharmacokinet 2001; 40: 375-82.
EMIT: EMIT refers to “Enzyme multiplied immunoassay technique”. This technique is a method for qualitative and quantitative determination of therapeutic and recreational drugs and certain proteins in serum and urine.
Envarsus®: brand name of a drug (marketed as Envarsus XR in United States by Veloxis Pharmaceuticals and marketed in Europe by Chiesi) comprising tacrolimus and given once daily (OAD).
FPIA: FPIA refers to “Fluorescence polarization immunoassay” and is a class of in vitro biochemical test used for rapid detection of antibody or antigen in sample.
GGally package: Please refer to the following Internet site: https://www.rdocumentation.org/packages/GGally/versions/1.5.0.
HPLC: HPLC refers to “High-performance liquid chromatography”. This technique is a technique in analytical chemistry used to separate, identify, and quantify each component in a mixture.
ISBA: the Immunosuppressant Bayesian Dose Adjustment (ISBA) website (www.pharmaco.chu-limoges.fr) allows to estimate the AUC of immunosuppressants by using maximum a posteriori Bayesian estimation (MAP-BE) on the basis of at least 3 blood samples and other characteristics such as age, delay post transplantation and method for drug measurement. (LCMS or HPLC for instance). In ISBA, each request posted is validated under 48 h by a trained pharmacologist, which represents a huge amount of time due to the large number of requests (>120000 since 2005).
LCMS: LCMS refers to” Liquid chromatography—mass spectrometry” andis an analytical chemistry technique that combines the physical separation capabilities of liquid chromatography (or HPLC) with the mass analysis capabilities of mass spectrometry (MS).
MAP-BE: Maximum a posteriori Bayesian estimation (technique used by ISBA)
MMF: Mycophenolate Mofetil® brand name of a drug (commercialized by Roche) prodrug of the mycophenolic acid
MPA: mycophenolic acid (MPA)
MPE: Mean Prediction Error
OAD: once a day
PALTP study: Riff, C., Debord, J., Monchaud, C., Marquet, P. & Woillard, J.-B. Population pharmacokinetic model and Bayesian estimator for 2 tacrolimus formulations in adult liver transplant patients (Br J Clin Pharmacol 85, 1740-1750, 2019).
PCCP study: defined in Benkali, K. et al. entitled “Tacrolimus population pharmacokinetic-pharmacogenetic analysis and Bayesian estimation in renal transplant recipients” (Clin Pharmacokinet 48, 805-816, 2009).
Pigrec study: defined in Fruit, D. et al. entitled “Ciclosporin population pharmacokinetics and Bayesian estimation in thoracic transplant recipients” (Clin Pharmacokinet 52, 277-288, 2013) and also in Woillard, J.-B. et al. entitled “Mycophenolic mofetil optimized pharmacokinetic modelling, and exposure-effect associations in adult heart transplant recipients” (Pharmacol. Res. 99, 308-315, 2015)
PK: pharmacokinetic profile
POPPK=Population pharmacokinetic
PKNCA: Denney [aut, B., cre, Buckeridge, C. & Duvvuri, S. PKNCA: Perform Pharmacokinetic Non-Compartmental Analysis. (2020). at <https://CRAN.R-project.org/package=PKNCA>
Prograf®: brand name of a drug (commercialized by Astellas Pharma) comprising tacrolimus and given twice daily (TAD).
RMSE: Root Mean Square Error
Stimmugrep study: it refers to the study that can be found in the article by Monchaud C, Rousseau A, Leger F, David O J, Debord J, Dantoine T, Marquet P. Limited sampling strategies using Bayesian estimation or multilinear regression for cyclosporin AUC(0-12) monitoring in cardiac transplant recipients over the first year post-transplantation. Eur J Clin Pharmacol. 2003 April; 58(12):813-20
TAD: twice a day
Tidyverse framework: The tidyverse is an opinionated collection of R packages designed for data science. It is available on www.tidyverse.org.
Xgboost: This method is described in the article T. Chen et al. entitled “XGBoost: A Scalable Tree Boosting System” (arXiv:1603.02754v3) wherein the definition and use of the parameters mtry, min_n, tree_depth, and learning_rate (Shrinkage in the article).
   The method was implemented with the R package that can be accessed at the following address: https://cran.rproject.org/web/packages/xgboost/vignettes/xgboostPresentation.html

1. A method for assessing the area-under-the-concentration-over-time value at a specific time of an immunosuppressant in blood or plasma of a subject after administration of a dose of the immunosuppressant, the subject being treated by a treatment comprising administrations of the drug, the method being computer-implemented and the method comprising the steps of:
providing parameters, the provided parameters comprising parameters relative to the treatment, and
applying a predicting function to the provided parameters to obtain area-under-the-concentration-over-time value at a specific time of the immunosuppressant, the predicting function being obtained by an artificial intelligence technique.

2. The method for assessing according to claim 1, wherein the intelligence artificial technique does not comprise using a neural network.

3. The method for assessing according to claim 1, wherein the intelligence artificial technique comprises using a gradient boosting technique, preferably an extreme gradient boosting technique.

4. The method for assessing according to claim 1, wherein the parameters relative to the treatment comprise several values of concentration of the immunosuppressant at different times after administration of the drug.

5. The method for assessing according to claim 1, wherein the parameters relative to the treatment comprise the values of difference in concentration of the immunosuppressant at a first given time and a second given time after administration of the drug for several couples of first given time and second given time.

6. The method for assessing according to claim 1, wherein the provided parameters further comprise at least one parameter relative to the subject.

7. The method for assessing according to claim 1, wherein the parameters relative to the treatment comprise the dose administered.

8. The method for assessing according to claim 1, wherein the treatment includes a transplantation of an organ to the subject, the parameters relative to the treatment comprising the delay between a request of assessment of the area-under-the-concentration-over-time curve of an immunosuppressant and the transplantation.

9. The method for assessing according to claim 1, wherein the provided parameters consist in the parameters relative to the treatment.

10. The method for assessing according to claim 1, wherein the immunosuppressant is a calcineurin inhibitor.

11. The method for assessing according to claim 1, wherein the immunosuppressant is an inosine monophosphate deshydrogenase inhibitor.

12. The method for assessing according to claim 1, wherein the immunosuppressant is a m-TOR inhibitor.

13. A method for monitoring patients to provide a quantitative measure for the therapeutic efficacy of a therapy by carrying out the steps of a method for assessing on said patients, the method for assessing being according to claim 1.

14. Computer program product comprising computer program instructions, the computer program instructions being loadable into a data-processing unit and adapted to cause execution of the method according to claim 1 when run by the data-processing unit.

15. Computer-readable medium comprising computer program instructions which, when executed by a data-processing unit, cause execution of the method according to claim 1.

16. The method for assessing according to any one of claim 1, wherein the immunosuppressant is tacrolimus.

17. The method for assessing according to any one of claim 1, wherein the immunosuppressant is ciclosporin.

18. The method for assessing according to any one of claim 1, wherein the immunosuppressant is sirolimus.

19. A method for assessing the area-under-the-concentration-over-time value at a specific time of an inhibitor in blood or plasma of a subject after administration of a dose of the inhibitor,
the inhibitor being a calcineurin inhibitor, a m-TOR inhibitor or a an inosine monophosphate deshydrogenase inhibitor,
the method comprising:
administrating the drug to the subject in a treatment of the subject,
obtaining:
several values of concentration of the inhibitor at different times after administration of the drug corresponding to a first parameter, and
the values of difference in concentration of the inhibitor at a first given time and a second given time after administration of the drug for several couples of first given time and second given time, corresponding to a second parameter, and
applying an extreme gradient boosting technique to at least the first and second parameters to obtain area-under-the-concentration-over-time value at a specific time of the inhibitor.

20. A method for assessing according to claim 19, wherein, the treatment includes a transplantation of an organ to the subject and during the step of obtaining, other parameters are obtained, among which:
the dose administered,
the delay between a request of assessment of the area-under-the-concentration-over-time curve of the inhibitor and the transplantation, and
the age of the subject.
